(19) United States
(12) Patent Application Publication
(10) Pub. No.: US 2011/0274624 A1
(43) Pub. Date: Nov. 10, 2011

(54) CONTRAST AGENTS IN POROUS PARTICLES

(75) Inventors: Paolo Decuzzi, Houston, TX (US); Lon J. Wilson, Houston, TX (US); Mauro Ferrari, Houston, TX (US); Jeyarama S. Ananta, Houston, TX (US); Biana Godin, Houston, TX (US)

(73) Assignees: WILLIAM MARSH RICE UNIVERSITY, Houston, TX (US); BOARD OF REGENTS OF THE UNIVERSITY OF TEXAS SYSTEM, Austin, TX (US)

(21) Appl. No.: 13/084,234

(22) Filed: Apr. 11, 2011

Related U.S. Application Data
(60) Provisional application No. 61/322,766, filed on Apr. 9, 2010.

(51) Int. Cl. A61K 49/06; A61P 43/00; A61K 49/18; A61K 49/08; B82Y 5/00

(52) U.S. Cl. 424/9.32; 424/9.3; 977/930

(57) ABSTRACT

MRI imaging compositions are disclosed comprising non-chelated MRI contrast agents in the pores of at least one porous microparticle or nanoparticle. The compositions of the invention have been found to exhibit increased relaxivity and therefore, enhanced MRI imaging. The non-chelated contrast agents include T1 contrast agents, such as those including Gd(III) or Mn(II). Methods of MRI imaging and methods of making the compositions are also disclosed.

### CROSS-REFERENCE TO RELATED APPLICATIONS

This application claims priority to U.S. Provisional Patent Application No. 61/322,766, filed on Apr. 9, 2010, the entirety of which is incorporated herein by reference.

### STATEMENT REGARDING FEDERALLY SPONSORED RESEARCH

This invention was made with government support under Grant Nos. W911NF-09-1-0044, W81XWH-09-1-0212, and W81XWH-09-2-0139 awarded by the Department of Defense; under Grant No. NNJ06HE06A awarded by the National Aeronautics and Space Administration; and under Grant Nos. CA128797 and CA143837 awarded by the National Institutes of Health. The government has certain rights in the invention.

### BACKGROUND OF THE INVENTION

Chemical contrast agents (CAs) have been widely used for improving the sensitivity and efficacy of various imaging systems, including magnetic resonance imaging (MRI). Despite their sensitivity and efficacy, the use of CAs for imaging suffer from various limitations, including low circulation time, insufficient contrast generation and potential toxicity. Therefore, there is currently a need to design new CA systems that address the aforementioned limitations.

### BRIEF SUMMARY OF THE INVENTION

The present invention, in one embodiment, is directed to a composition comprising at least one porous microparticle or nanoparticle and at least one non-chelated MRI contrast agent in pores of the least one porous microparticle or nanoparticle. The porous microparticles or nanoparticles may comprise at least one nanoporous microparticle or nanoparticle and may vary in pore size. For example, the pore size may range from 5 nm to 200 nm, from 10 nm to 100 nm, or have a maximum dimension of no more than 10 microns. The microparticles or nanoparticles may also have different shapes, including non-spherical shapes. In other embodiments, the shape of the porous microparticles or nanoparticles may be a hemispherical particle, a quasi-hemispherical particle, or a discoidal particle.

The compositions of the present invention may also comprise a plurality of particles that are uniform in size. The compositions may further comprise a pharmaceutically acceptable carrier, where the porous microparticles or nanoparticles are suspended in said carrier. The compositions of the present invention may also comprise at least one silicon or silica porous microparticle or nanoparticle.

In various embodiments, the non-chelated MRI contrast agent may be a non-chelated T1 MRI contrast agent. The MRI contrast agent may comprise at least one of Gd(III) or Mn(II). In one embodiment, the non-chelated T1 MRI contrast agent comprises Gd(III). The Gd(III) contrast agent includes any Gd(III)-based contrast agent, including gadobenic acid, gadobutrol, gadocoletic acid, gadodenterate, gadodiamide, gadofosveset, gadomelitol, gadopenamide, gadopentetic acid, gadoteric acid, gadoversetamide, gadoxetic acid or a pharmaceutically acceptable salt thereof. In another embodiment, the contrast agent comprises gadopentetic acid or a pharmaceutically acceptable salt thereof. In some embodiments, the non-chelated T1 MRI contrast agent comprises at least one carbon based particle comprising Gd(III), such as gadofullerene or gadonanotube. The gadonanotube may be bundled or unbundled.

The present invention, in other embodiments, is directed to a method of MRI imaging by administering to a subject in need thereof an effective amount of a composition comprising: (1) at least one porous microparticle or nanoparticle; and (2) at least one non-chelated MRI contrast agent in pores of the least one porous microparticle or nanoparticle. In various embodiments, the method may also comprise detecting a signal from the subject associated with the at least one non-chelated MRI contrast agent. The administration may, in some embodiments, be performed intravascularly. The method may be conducted on any patient, including a human being.

The method may utilize nanoporous microparticles or nanoparticles having varying pore sizes. For example, the pore size may be from 5 nm to 200 nm, from 10 nm to 100 nm, or have a maximum dimension of no more than 10 microns. The shape of the microparticles or nanoparticles may be non-spherical, such as hemispherical, quasi-hemispherical, or discoidal.

The method may also utilize a plurality of the porous microparticles or nanoparticles that are uniform in size. In some embodiments, the compositions may further comprise a pharmaceutically acceptable carrier, where the porous microparticles or nanoparticles are suspended in the carrier.

In some embodiments, the particles may comprise silicon or silica. The non-chelated MRI contrast agent may be a T1 MRI contrast agent, such as one comprising Gd(III) or Mn(II). The Gd(III) contrast agent may include gadobenic acid, gadobutrol, gadocoletic acid, gadodenterate, gadodiamide, gadofosveset, gadomelitol, gadopenamide, gadopentetic acid, gadoteric acid, gadoversetamide, gadoxetic acid or a pharmaceutically acceptable salt thereof. In some embodiments, the non-chelated T1 MRI contrast agent comprises gadopentetic acid or a pharmaceutically acceptable salt thereof. In other embodiments, the non-chelated T1 MRI contrast agent comprises at least one carbon based particle comprising Gd(III), such as gadofullerene or gadonanotube. The gadonanotube may be bundled or unbundled.

The present invention is further directed to a method of making a composition for MRI imaging. Such methods generally comprise exposing at least one porous microparticle or nanoparticle to a solution comprising at least one non-chelated MRI contrast agent. The exposing generally leads to the loading of the at least one non-chelated MRI contrast agent into pores of the least one porous microparticle or nanoparticle. This method may further comprise drying the at least one porous microparticle or nanoparticle prior to the exposing. Moreover, the exposing step may comprise: (1) exposing the at least one porous microparticle or nanoparticle to a first solution comprising the at least non-chelated MRI contrast agent; (2) washing the exposed porous microparticle or nanoparticle (3) and exposing the washed microparticle or nanoparticle to a second solution comprising the at least one non-chelated MRI contrast agent. In other embodiments, the method may further comprise: (4) washing the loaded porous microparticle or nanoparticle after said exposing; and/or (5) sonicating the particles during said exposing. The particles may comprise at least one silicon porous microparticle or nanoparticle, which may optionally be oxidized silicon porous microparticles or nanoparticles. In some embodiments, the non-chelated MRI contrast agent comprises at least one non-chelated T1 MRI contrast agent, such as those comprising Gd(III) or Mn(II). The Gd(III) MRI contrast agents include, for example, gadobenic acid, gadobutrol, gadocoletic acid, gadodenterate, gadodiamide, gadofosveset, gadomelitol, gadopenamide, gadopentetic acid, gadoteric acid, gadoversetamide, gadoxetic acid or a pharmaceutically acceptable salt thereof. In another embodiment, the MRI contrast agent may be gadopentetic acid or a pharmaceutically acceptable salt thereof. The invention further encompasses the use of a non-chelated T1 MRI contrast agent comprising at least one carbon based particle comprising Gd(III), such as gadofullerene or gadonanotube. The gadonanotube may be bundled or unbundled.

### BRIEF DESCRIPTION OF THE FIGURES

In order that the manner in which the above recited and other advantages and objects of the invention are obtained, a more particular description of the invention briefly described above will be rendered by reference to specific embodiments thereof, which are illustrated in the appended Figures. Understanding that these Figures depict only typical embodiments of the invention and are therefore not to be considered limiting of its scope, the invention will be described with additional specificity and detail through the use of the accompanying Figures in which:

FIG. 1 schematically represents embodiments of the compositions of the present invention (as MRI nanoconstructs in this embodiment). Magnevist (MAG) (FIG. 1A) and debundled gadonanotubes (GNT) (FIG. 1B) are loaded into mesoporous silicon particles (SiMPs) with different sizes and shapes. Scanning electron micrographs (SEMs) of the (FIG. 1C) quasi-hemispherical (H-SiMP: 1.6 μm in diameter and 1.0 μm in thickness) and (FIG. 1D) discoidal (D-SiMP: 1.0 μm in diameter and 0.4 μm in thickness) particles are shown. FIG. 1E shows illustrations of MAG and GNT entrapped within the porous structure of the SiMPs. The geometrical confinement of the Gd-based contrast agent (CA) within the nanopores of the SiMPs enhances the T₁ contrast by altering both the inner- and outer-sphere contributions.

FIG. 2 provides the concentration of Gd³⁺ ion in the SiMP nanoconstruct as determined by ICP-OES analysis. Comparison between the two loading procedures used in the study are included: (i) single-step loading (gray columns) and (ii) sequential loading (black columns). Two different aqueous stock solutions with GNT (pluronic surfactant) were exposed to the nanoporous SiMPs, namely 200 and 300 μl. No statistically significant difference has been observed between the two loading procedures.

FIG. 3 provides SEMs of the H-SiMPs loaded with GNTs. The GNTs (coated with pluronic surfactant) could be seen adhering to the lateral surface of the pore walls and are quite uniformly distributed over the whole field of view (sequential loading experiments). The front (FIG. 3A) and back (FIG. 3B) of the SiMPs are shown. MAG and GF are too small to be captured through a SEM image.

FIG. 4 represents the MRI characterization of the nanoconstruct in a bench-top relaxometer. The longitudinal relaxivity r₁ of the four new MRI nanoconstructs is compared with the corresponding Gd-based CAs presented in a bar chart (FIG. 4A) and in tabular form (FIG. 4B) (1.41 T and 37° C.). Data are presented as mean±SD (n≧4).

FIG. 5 represents the MRI characterization of the H-SiMP/GNT nanoconstruct in a clinical scanner. FIG. 5A shows that the inversion recovery fit for SiMPs and SiMP/GNT nanoconstructs were acquired using an inversion recovery pulse sequence and plotted as a function of their inversion time Tᵢₙᵥ (time at which the signal is completely suppressed). FIG. 5B shows inversion recovery phantoms for SiMP and SiMP/GNT nanoconstruct, showing faster recovery for the nanoconstruct. Data were obtained using a 1.5 T commercial clinical scanner with TR=7500 ms and TE=20 ms.

FIG. 6 provides the calculated longitudinal relaxivity for the SiMP/MAG nanoconstruct. The experimental NMRD profile for Magnevist (dots)⁶ is compared with three curves (solid lines) derived from the Solomon, Bloembergen and Morgan (SBM) Theory for different values of the parameters (FIG. 6A) τ_{R}(=54, 270 and 540 ps) and (FIG. 6B) τ_{D}(=40, 180 and 400 ps). FIG. 6C shows the calculated maximum longitudinal relaxivity r₁ of the SiMP/MAG nanoconstructs as a function of the governing parameters τ_{R} and τ_{D}. All the other parameters are from Table 1 for Magnevist. FIG. 6D shows the magnetic properties of Magnevist, as derived from the best fitting of the experimental NMRD.

FIG. 7 provides NMRD profiles for the GNT and the SiMP/GNT construct. FIG. 7A shows the experimental NMRD profile for debundled GNTs (as GadoDex; dotted lines)⁹, which is compared with three best fitting curves (solid lines) derived from the SBM Theory for different values of the parameters q and τₘ. The experimental NMRD profiles for debundled GNTs (as GadoDex; dotted line) and simulated curves derived from the SBM Theory (FIG. 7B) for different values of q, namely, q=2, 4 and 6 and (FIG. 7C) different values of τₘ, namely, 0.1, 1.5 and 2.9 ns. All other parameters are from Table 1 for the CNTs. FIG. 7D shows magnetic properties of debundled GNTs, as derived from the best fitting of the experimental NMRD.

FIG. 8 provides the variation of the relaxation rate (1/T₁-1/T_{1d}) as a function of the concentration of Gd³⁺ ions within the SiMPs. The experimental results (solid points) are quite closely aligned along a straight line.

FIG. 9 provides the NMRD profile for Gd-DTPA (Magnevist). Comparison between the predictions of SBM Theory (--- inner-sphere contribution only; ______ inner- and outer-sphere contributions) and the experimental data (solid circles) are shown. The parameters used for the simulated curves are listed in Table 1.

FIG. 10 shows the NMRD profile for Gd-DOTA (Dotarem). Comparison between the predictions of SBM Theory (- - - inner-sphere contribution only; — inner- and outer-sphere contributions) and the experimental data (solid circles) are shown. The parameters used for the theoretical curves are listed in Table 1.

FIG. 11 demonstrates the effect of changing the characteristic time for splitting, τ_{ν}, on the theoretically-derived NMRD profile (τ_{ν}=0.01, 20, 40, 60 ps). Significant differences are observed only as τ_{ν} decreases below 10 ps. All other parameters are from Table 1.

FIG. 12 depicts the effect of changing the mean square zero field splitting energy (ZFS) Δ² on the theoretically derived NMRD profile (Δ²=0, 5, 10, 15 and 20×10⁺¹⁸ s⁻²). As Δ² reduces, the relaxivity reduces and the maxima at high field strength move towards larger frequencies. All other parameters are from Table 1.

FIG. 13 represents the contribution of the Outer Sphere to the estimated r₁ relaxivity (as derived from SBM Theory) as a function of τₘ. The contributions for different values of the field strength are shown. The field strengths range between ν_{I}=20 and 120 MHz. All other parameters are from Table 1.

FIG. 14 depicts the estimated longitudinal relaxivities for debundled GNTs (as GadoDex). The data are derived from the SBM Theory as a function of τₘ for different values of q that range between 2 and 8 (FIG. 14A), and for different values of the magnetic field ν₁ that range between 20 and 100 MHz (FIG. 14B). All other parameters are from Table 1.

FIG. 15 is a transmission electron micrograph (TEM) image of Dextran-coated Gadonanotubes (GadoDex).

FIG. 16 shows SEMs of D-SiMPs loaded with GNTs. The GNTs appear as particles adhered to the walls of the pores that are quite uniformly distributed within the field of view.

FIG. 17 shows a comparison of longitudinal relaxivity (r1) data of various MAG-loaded mesoporous silicon particles. The particles loaded included HP particles (pore sizes of about 30-40 nm in diameter), SP particles (pore sizes of about 5-10 nm in diameter), and GP particles (pore sizes of less than about >50 nm in diameter).

### DETAILED DESCRIPTION OF EXEMPLARY EMBODIMENTS

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

### RELATED APPLICATIONS AND PUBLICATIONS

The following research articles and patent documents, which are all incorporated herein by reference in their entirety, may be useful for understanding the present invention: (1) PCT Publication No. WO2007/120248, published Oct. 25, 2007; (2) PCT Publication No. WO2008/041970, published Apr. 10, 2008; (3) PCT Publication No. WO2008/021908, published Feb. 21, 2008; (4) U.S. Patent Application Publication No. 2008/0102030, published May 1, 2008; (5) U.S. Patent Application Publication No. 2003/0114366, published Jun. 19, 2003; (6) U.S. Patent Application Publication No. 2008/0206344, published Aug. 28, 2008; (7) U.S. Patent Application Publication No. 2008/0280140, published Nov. 13, 2008; (8) PCT Patent Application PCT/US2008/014001, filed Dec. 23, 2008; (9) U.S. Pat. No. 6,107,102, issued Aug. 22, 2000; (10) U.S. Patent Application Publication No. 2008/0311182, published Dec. 18, 2008; (11) PCT Patent Application PCT/US2009/000239, filed Jan. 15, 2009; (12) PCT Patent Application PCT/US11/27746, filed Mar. 9, 2011; (13) U.S. Patent Application Publication No. 2010/0029785, published Feb. 4, 2010; and (14) Tasciotti E. et al, 2008 Nature Nanotechnology 3:151-157.

### DEFINITIONS

Unless otherwise specified “a” or “an” means one or more.

“Microparticle” means a particle having a maximum characteristic size from 1 micron to 1000 microns, or from 1 micron to 100 microns. “Nanoparticle” means a particle having a maximum characteristic size of less than 1 micron.

“Nanoporous” or “nanopores” refers to pores with an average size of less than 1 micron.

“Biodegradable material” refers to a material that can dissolve or degrade in a physiological medium, such as PBS or serum.

“Biocompatible” refers to a material that, when exposed to living cells, will support an appropriate cellular activity of the cells without causing an undesirable effect in the cells such as a change in a living cycle of the cells; a release of proinflammatory factors; a change in a proliferation rate of the cells and a cytotoxic effect.

“Contrast agent” refers to a moiety that increases the contrast of a tissue or structure being examined. This agent may be used to increase the contrast of the tissue or structure being examined using magnetic resonance imaging (MRI), optimal imaging, or a combination thereof. The moiety can be part of a specific part of or a whole molecule or part of a hybrid delivery system.

Physiological conditions stand for various conditions, such as temperature, osmolarity, pH and motion close to that of plasma in a mammal body, such as a human body in the normal state.

Introduction

Magnetic resonance imaging (MRI) has evolved into one of the most powerful, non-invasive diagnostic imaging techniques in medicine and biomedical research. The optimal resolution and in-depth anatomical details provided by MRI are useful for early diagnosis of many diseases. The nuclear spin of water protons, which are present in abundance in the body, is manipulated by external magnetic fields in MRI to obtain images. In the magnetization of water protons, two characteristic relaxation times are mainly considered: the longitudinal T₁ and transverse T₂ relaxation times. The values of these relaxation times are tissue dependent, which allows for the generation of contrast.

Chemical contrast agents (CAs) have been widely used for improving the sensitivity and diagnostic confidence in MRI. In 2007, there were about 28 million MRI procedures performed in the United States, and nearly 45% of them used CAs as part of the imaging procedure. These CAs contain paramagnetic metal ions, such as gadolinium or manganese ions, that exhibit time-dependent magnetic dipolar interaction with the surrounding water protons and improve the MRI sensitivity by decreasing the relaxation time T₁ of water protons. The most widely-used clinical CAs use gadolinium ions (Gd³⁺) as the paramagnetic ion.

In spite of the enormous progress achieved in the design and synthesis of clinical MRI CAs, the current agents suffer from several limitations including low circulation time, insufficient contrast generation and potential toxicity (Nephrogenic Systemic Fibrosis). In particular, naked (aqueous) Gd³⁺ ions are toxic. Therefore, for biological use, such ions need to be sequestered through the use of a variety of linear and macrocyclic chelates.²⁻⁴ Chelation minimizes the toxicity of the paramagnetic ions as long as they are not released by demetallation in the circulation, or by transmetallation with other ions present in the body (e.g., Zn²⁺). However, at the same time, chelation decreases the number of coordination sites available for water proton exchange (e.g., 8-9 sites for free Gd³⁺, as compared to 1-2 sites for Gd³⁺-chelate compounds). This results in reduced contrast enhancement (relaxivity).

In addition, almost all of the clinically-used CAs are extracellular fluid (ECF) space agents with low blood circulation times (few minutes) and minimal tissue selectivity and cellular uptake. Such attributes limit the CAs' contrast enhancement even more. Moreover, clinically-used CAs have r₁ relaxivities smaller than 4 mM⁻¹ s⁻¹ at 1.41 T, as listed in Table 1. Thus, in view of the aforementioned limitations, there is an important need to design new MRI CA systems with optimal performance and more desirable physiochemical properties with the aim of enhancing their detection limits (e.g., potentially to the single cell level).

The present invention relates to delivery systems and compositions comprising: (1) porous microparticles or nanoparticles; and (2) non-chelated MRI contrast agents loaded into the pores of the nanoparticles or microparticles. The contrast agent may be a T1 MRI contrast agent, such as a gadolinium (Gd(III))-based or a manganese (Mn(II))-based contrast agent. This delivery system may provide substantially increased relaxivity (rᵢ) relative to the naked contrast agent, thereby leading to enhanced optical imaging. Typically, chelating agents, molecules, molecular ions, or species having an unshared electron pair for donation to a metal ion are used with various contrast agents (e.g., Gd(III) and/or (MN(II)). Such molecules reduce the contrast agents' toxicity to the patient. The molecules also reduce the number of coordination sites available for water proton exchange with the contrast agents. The present invention, on the other hand, eliminates the need for chelation of the contrast agents (e.g., Gd-CAs and Mn-CAs). This in turn helps obtain enhanced relaxivity and optical imaging from the contrast agents.

Additional aspects of the present invention relate to methods of making the above-mentioned compositions and delivery systems. Further embodiments of the present invention pertain to methods of MRI imaging by administering the compositions of the present invention to a subject. Additional details regarding various aspects of the present invention will now be discussed in more detail as specific and non-limiting embodiments.

Contrast Agents

The present invention has demonstrated enhanced efficiency of chemical contrast agents by confining them within microparticles or nanoparticles. Any chemical contrast agent for optical imaging systems, such as MRI systems, may be used with the present invention. In one embodiment, the contrast agent is a T1 MRI contrast agent. In another embodiment, the contrast agent is a Gd(III)-based contrast agent (Gd-CA), including, but not limited to, gadobenic acid, gadobutrol, gadocoletic acid, gadodenterate, gadodiamide, gadofosveset, gadomelitol, gadopenamide, gadopentetic acid, gadoteric acid, gadoversetamide, gadoxetic acid or a pharmaceutically acceptable salt thereof. In another embodiment, the contrast agent may also comprise the Gd(III) ion in a carbon-based particle. For example, the Gd(III)-CA may comprise a gadofullerene or a gadonanotube. Moreover, the gadonanotube may be bundled or unbundled. In another embodiment, the contrast agent is a Mn(II)-based contrast agent (Mn-CA), as known in the art. In some embodiments, the contrast agent may comprise Magnevist (MAG) and/or Dotarem (See Examples 1-2 below). In further embodiments, the contrast agents may be pharmaceutically acceptable salts of the above-mentioned contrast agents.

In some embodiment, the contrast agents of the present invention are not chelated (i.e., non-chelated). Without being bound by theory, it is envisioned that avoiding chelation allows the contrast agents to expose more coordination sites for the water molecules and increase the relaxivity of the contrast agents. Decreasing the movement of the water molecules can therefore enhance the MRI imaging. In other embodiments, the contrast agents may optionally be chelated. In another embodiment, a combination of chelated and non-chelated contrast agents may optionally be used. The use of additional contrast agents in the compositions of the present invention that have not been disclosed here can also be envisioned by persons of ordinary skill in the art.

In various embodiments, the contrast agents of the present invention can also comprise an additional functional moiety or moieties directed toward detection of a particular disease or for imaging a particular tissue, organ, cell, or other structure. Such moieties, as known in the art, may include a targeting moiety directed to the delivery of the agent to the desired tissue, cell type, or structure. Therefore, the targeting moiety can cause the contrast agent within the pores of the nanoparticles or microparticles to concentrate in the targeted tissue, cell type or structure, such as cancer cells or tumors.

Non-limiting examples of targeting moieties include, without limitation, antibodies, aptamers, and small molecules. Additional examples of targeting moieties are disclosed in US Patent Application Publication No. 2008/0311182.

Porous Microparticles or Nanoparticles

The porous microparticles and nanoparticles of the present invention may be loaded and/or encapsulated with one or more contrast agents. In the case of multistage delivery systems, the porous microparticles or nanoparticles of the present invention may be loaded and/or encapsulated with second stage particles, which may contain one or more active agents.

The porous microparticles or nanoparticles of the present invention may also have a variety of shapes and sizes (hereinafter “particles” or “delivery systems”). The dimensions of the particles are not particularly limited and may depend on a particular application. For example, for intravascular administration, a maximum characteristic size of the particle may be smaller than a radius of the smallest capillary in a subject, which is about 4 to 5 microns for humans. In some embodiments, the maximum characteristic size of the particle may be less than about 100 microns, less than about 50 microns, less than about 20 microns, less than about 10 microns, less than about 5 microns, less than about 4 microns, less than about 3 microns, less than about 2 microns, or less than about 1 micron. Yet, in some embodiments, the maximum characteristic size of the particles may be from 100 nm to 3 microns, from 200 nm to 3 microns, from 500 nm to 3 microns, or from 700 nm to 2 microns. In more specific embodiments, the maximum characteristic size of the particle may be greater than about 2 microns, greater than about 5 microns, or greater than about 10 microns.

The shape of the particle is not particularly limited. In some embodiments, the particle may be a spherical particle. Yet, in some embodiments, the particle may be a non-spherical particle. In some embodiments, the particle can have a symmetrical shape. Yet, in some embodiments, the particle may have an asymmetrical shape.

In some embodiments, the particle may have a selected non-spherical shape configured to facilitate a contact between the particle and a surface of the target site, such as an endothelium surface of the inflamed vasculature. Examples of appropriate shapes include, but are not limited to, an oblate spheroid, hemispherical, quasi-hemispherical, a disc or a cylinder. In some embodiments, the particle may be such that only a portion of its outer surface defines a shape configured to facilitate a contact between the particle and a surface of the target site, such as endothelium surface, while the rest of the outer surface does not. For example, the particle can be a truncated oblate spheroidal particle.

The dimensions and shapes of particles that may facilitate a contact between the particles and a surface of the target site may be evaluated using various methods. Non-limiting examples of such methods are disclosed in US Patent Application Publication Nos. 2008/0206344 and 2010/0029785.

In some embodiments, the particles to be modified with an isolated cellular membrane may be a porous particle (i.e., a particle that comprises a porous material). The porous material may be a porous oxide material or a porous etched material. Examples of porous oxide materials include, but are no limited to, porous silicon microparticles or nanoparticles (e.g., porous silicon oxide), porous silica microparticles or nanoparticles, porous aluminum oxide, porous titanium oxide and porous iron oxide. The term “porous etched materials” refers to a material in which pores are introduced via a wet etching technique, such as electrochemical etching or electroless etching. Examples of porous etched materials include porous semiconductors materials, such as porous silicon, porous germanium, porous GaAs, porous InP, porous SiC, porous SiₓGe₁₋ₓ, porous GaP and porous GaN. Methods of making porous etched particles are disclosed, for example, in US Patent Application Publication No. 2008/0280140.

In some embodiments, the porous particles may be nanoporous particles. In further embodiments, the porous particles may be mesoporous particles. In some embodiments, an average pore size of the porous particles may be from about 1 nm to about 1 micron, from about 1 nm to about 800 nm, from about 1 nm to about 500 nm, from about 1 nm to about 300 nm, from about 1 nm to about 200 nm, or from about 2 nm to about 100 nm. In additional embodiments, the average pore size of the porous particles can be no more than 10 microns, no more than 1 micron, no more than 800 nm, no more than 500 nm, no more than 300 nm, no more than 200 nm, no more than 100 nm, no more than 80 nm, or no more than 50 nm. In some embodiments, the average pore size of the porous particles can be a size from about 5 nm to about 100 nm, from about 5 nm to about 200 nm, from about 10 nm to about 60 nm, from about 10 nm to about 100 nm, from about 20 nm to about 40 nm, or from about 30 nm to about 10 nm. In some embodiments, the average pore size of the porous particles can be from about 1 nm to about 10 nm, from about 3 nm to about 10 nm, or from about 3 nm to about 7 nm.

In some embodiments, the particles may comprise a plurality of particles that have uniform pore sizes. Yet, in some embodiments, the particles may comprise a plurality of particles that have different pore sizes.

In general, pores sizes may be determined using a number of techniques, including N₂ adsorption/desorption and microscopy, such as scanning electron microscopy (SEM). In some embodiments, pores of the porous particles may be linear pores. Yet, in some embodiments, pores of the porous particles may be sponge like pores.

Methods of loading active agents into porous particles are disclosed, for example, in U.S. Pat. No. 6,107,102 and US Patent Application Publication No. 2008/0311182. In some embodiments, after the contrast agent is loaded, the pores of the porous particles may sealed or capped.

In some embodiments, at least a portion of the porous particles may comprise a biodegradable region. In many embodiments, the whole particle may be biodegradable.

In some embodiments, the particles may comprise silicon or silica. In general, porous silicon may be bioinert, bioactive or biodegradable, depending on its porosity and pore size. Also, a rate or speed of biodegradation of porous silicon may depend on its porosity and pore size, see e.g. Canham, Biomedical Applications of Silicon, in Canham L T, editor. Properties of porous silicon. EMIS datareview series No. 18. London: INSPEC. p. 371-376. The biodegradation rate may also depend on surface modification.

Porous silicon particles and methods of their fabrication are disclosed, for example, in Cohen M. H. et al. Biomedical Microdevices 5:3, 253-259, 2003; US Patent Application Publication No. 2003/0114366; U.S. Pat. Nos. 6,107,102 and 6,355,270; US Patent Application Publication No. 2008/0280140; PCT Publication No. WO 2008/021908; Foraker, A. B. et al. Pharma. Res. 20 (1), 110-116 (2003); and Salonen, J. et al. Jour. Contr. Rel. 108, 362-374 (2005). Porous silicon oxide particles and methods of their fabrication are disclosed, for example, in Paik J. A. et al., J. Mater. Res., Vol. 17, August 2002, p. 2121.

In some embodiments, the particles may comprise a biodegradable material. For oral administration, such material may be a material designed to erode in the GI tract. In some embodiments, the biodegradable particle may be formed of a metal, such as iron, titanium, gold, silver, platinum, copper, and alloys and oxides thereof. In some embodiments, the biodegradable material may be a biodegradable polymer, such as polyorthoesters, polyanhydrides, polyamides, polyalkylcyanoacrylates, polyphosphazenes, and polyesters. Exemplary biodegradable polymers are described, for example, in U.S. Pat. Nos. 4,933,185, 4,888,176, and 5,010,167. Specific examples of such biodegradable polymer materials include poly(lactic acid), polyglycolic acid, polyglycolic-lactice acid (PGLA); polycaprolactone, polyhydroxybutyrate, poly(N-palmitoyl-trans-4-hydroxy-L-proline ester) and poly(DTH carbonate).

The particles may be prepared using a number of techniques. In some embodiments, the particles of the delivery system may be a particle produced utilizing a top-down microfabrication or nanofabrication technique, such as photolithography, electron beam lithography, X-ray lithography, deep UV lithography, nanoimprint lithography or dip pen nanolithography. Such fabrication methods may allow for a scaled up production of particles that are uniform or substantially identical in dimensions.

In some embodiments, the delivery system may be a multistage delivery system, which may comprise a larger first stage microparticle or nanoparticle, which may contain one or more smaller size second stage particles. Multistage delivery systems are disclosed, for example, in US Patent Application Publications Nos. 2008/0311182 and 2008/0280140, as well as in Tasciotti E. et al, 2008 Nature Nanotechnology 3, 151-157.

Various delivery systems, which may be used, are disclosed in PCT Publication Nos. WO 2008/041970 and WO 2008/021908; U.S. Patent Application Publications Nos. 2008/0102030, 2003/0114366, 2008/0206344, 2008/0280140, 2010/0029785, and 2008/0311182; PCT Patent Application PCT/US2008/014001, filed Dec. 23, 2008; PCT Patent Application PCT/US2009/000239, filed Jan. 15, 2009; U.S. Pat. Nos. 6,107,102 and 6,355,270; and PCT Patent Application PCT/US11/27746, filed Mar. 9, 2011.

Loading of Microparticles or Nanoparticles with Contrast Agents

Additional embodiments of the present invention pertain to methods of making the above-described delivery systems and compositions. Such methods generally comprise exposing at least one porous microparticle or nanoparticle to a solution comprising at least one non-chelated MRI contrast agent. Such exposure generally causes a loading of the non-chelated MRI contrast agents into the pores of the microparticles or nanoparticles. In some embodiments, the porous microparticles or nanoparticles are dried prior to the exposing. In various embodiments, the particles may be sonicated during the exposing step.

In some embodiments, the loading and confinement of CAs within the porous nanoparticles or microparticles occurs primarily through capillary forces. When the dry nanopores of the nanoparticles or microparticles are exposed to a concentrated aqueous solution of the CA, the latter are drawn within the pores due to capillary action. The loading of the CAs into the particles may be conducted in known manners.

In some embodiments, the CAs are loaded through single-step loading. In other embodiments, the CAs are loaded through sequential loading, where the nanoparticles or microparticles are exposed multiple times to a concentrated solution of CAs. For sequential loading, the nanoparticles or microparticles may be washed with HPLC grade water between each step. Thus, in some embodiments, the exposing step may comprise: (1) exposing the porous microparticle or nanoparticle to a first solution that contains the non-chelated MRI contrast agent; (2) washing the exposed porous microparticle or nanoparticle; and (3) exposing the washed microparticle or nanoparticle with a second solution that also contains the non-chelated MRI contrast agent.

Additional methods of loading CAs into microparticles or nanoparticles can also be envisioned. The amount of CAs within the loaded nanoparticles or microparticles may be determined by elemental analysis using inductively-coupled plasma optical emission spectrometry (ICP-OES).

Administration and Imaging

The delivery systems and compositions of the present invention may be administered to a subject (e.g., an animal or human) via a suitable administration methods in order to diagnose and/or monitor one or more physiological conditions (e.g., diseases). In some embodiments, a plurality of different compositions and/or delivery systems may be administered at the same time. In other embodiments, a plurality of the same compositions or delivery systems may be administered.

In various embodiments, the MRI imaging may be performed on a biological sample, including, but not limited to, a cell, a tissue, a structure, or a subject. In another embodiment, the imaging utilizes MRI or other optical imaging techniques.

In some embodiments, the invention is directed to methods of MRI imaging by: (1) administering to a subject in need of the imaging an effective amount of a composition or delivery system of the present invention that contains a non-chelated MRI contrast agent; and (2) detecting a signal from the subject that is associated with the non-chelated MRI contrast agent. A method of imaging may comprise administering to a subject a non-chelated contrast agent that is contained within the pores of a nanoparticle or a microparticle and rendering a magnetic resonance image of the subject. The amount administered is an effective amount sufficient to produce adequate imaging and will vary based on the particular subject, target, mode of administration, and desired effect. The particular administration method employed for a specific application may be determined by the attending physician. Typically, the composition may be administered by one of the following routes: topical; parenteral, including intravenous (i.v.), intramuscular (i.m.) and subcutaneous (s.c.) injection; inhalation, including pulmonary inhalation; oral; intraocular; intranasal; bucal; vaginal and anal.

In various embodiments, administration of the delivery system or composition may be systemic or local. In some embodiments, the administration of the delivery system or composition may be intravascular. The non-parenteral examples of administration recited above are examples of local administration. Intravascular administration can be either local or systemic. Local intravascular delivery can be used to bring a therapeutic substance to the vicinity of a known lesion by use of guided catheter system, such as a CAT-scan guided catheter, or by portal vein injection. General injection, such as a bolus i.v. injection or continuous/trickle-feed i.v. infusion are typically systemic.

In some embodiments, the composition containing the delivery system may be administered via i.v. infusion, via intraductal administration, or via an intratumoral route. The delivery systems may be formulated as a suspension that contains a plurality of them.

In some embodiments, individual delivery systems may be uniform in their dimensions and their content. To form the suspension, the delivery systems may be suspended in a suitable pharmaceutically acceptable carrier, such as an aqueous carrier vehicle. A suitable pharmaceutically acceptable carrier may be the one that is non-toxic to the recipient at the dosages and concentrations employed and is compatible with other ingredients in the formulation. Preparation of suspension of microfabricated particles is disclosed, for example, in US Patent Application Publication No. 2003/0114366.

Advantages

In one embodiment, the CAs loaded into the nanoparticles or microparticles may exhibit increased relaxivity relative to naked CAs. The ability of a paramagnetic material to act as an MRI contrast agent is expressed in terms of its relaxivity (rᵢ). This can be described as the change in the relaxation rate (1/Tᵢ; s⁻¹) of water protons per mM concentration of the CAs and can be calculated using the expression rᵢ=(1/Tᵢ-1/T_{id})/[CA], where Tᵢ is the relaxation time in the presence of the CA, T_{id} is the relaxation time in the absence of CA, and [CA] is the concentration of the CA (e.g., Gd³⁺ ion) present in solution (mM).

In one embodiment, the Gd-CA-loaded particles of the present invention may have an increase in rᵢ of at least 1.5 times, at least 2.0 times, at least 2.5 times, at least 3.0 times, at least 3.5 times, at least 4.0 times, at least 10 times, at least 18 times, at least 20 times, at least 30 times, or at least 40 times more than Gd-CA alone. While not wishing to be bound by theory, it is believed that the increased relaxivity observed can be attributed to the geometrical confinement of the Gd-CA molecules and their final organization within the pores. Geometrical confinement is believed to reduce the ability of the CAs to tumble; decrease the mobility of water molecules; and favor clustering and mutual interactions among the loaded CAs, thereby altering the original values of the governing parameters q, τₘ, τ_{R} and τ_{D}, and potentially others.

In some embodiments, the delivery systems of the present invention exhibit high r₁ values, but also constitute a formidable particle-based system for efficient intravascular delivery. The size, shape and surface properties of the particles (such as mesoporous silicon particles or SiMPs) can be rationally designed and tailored to enhance the accumulation of the contrast agents (such as T1 MRI) within a desired biological target site; to alter overall half-life in blood; and to control degradation. The delivery systems of the present invention could also play an important role in the development of single-cell imaging techniques, where high relaxivity (r₁>100 mM⁻¹ s⁻¹) and large localized Gd³⁺ concentration ([Gd³⁺]>10⁷/cell) are needed. Finally, these systems could be loaded with multiple agents, such as other nanoparticles, small molecules and drugs, to originate highly-multifunctional systems with imaging and therapeutic capabilities.

Embodiments described herein are further illustrated by, though in no way limited to, the following working examples.

### EXAMPLES

### Example 1

### Characterization of Gd-Based Contrast Agents

In this work, the enhanced efficiency of Gd-based CAs (Gd-CAs) has been demonstrated by confining them within the nanoporous structure of intravascularly injectable silicon particles (SiMPs)⁸. Enhanced efficiency was shown for two different Gd-CAs, namely Magnevist (MAG) and gadonanotubes (GNTs).

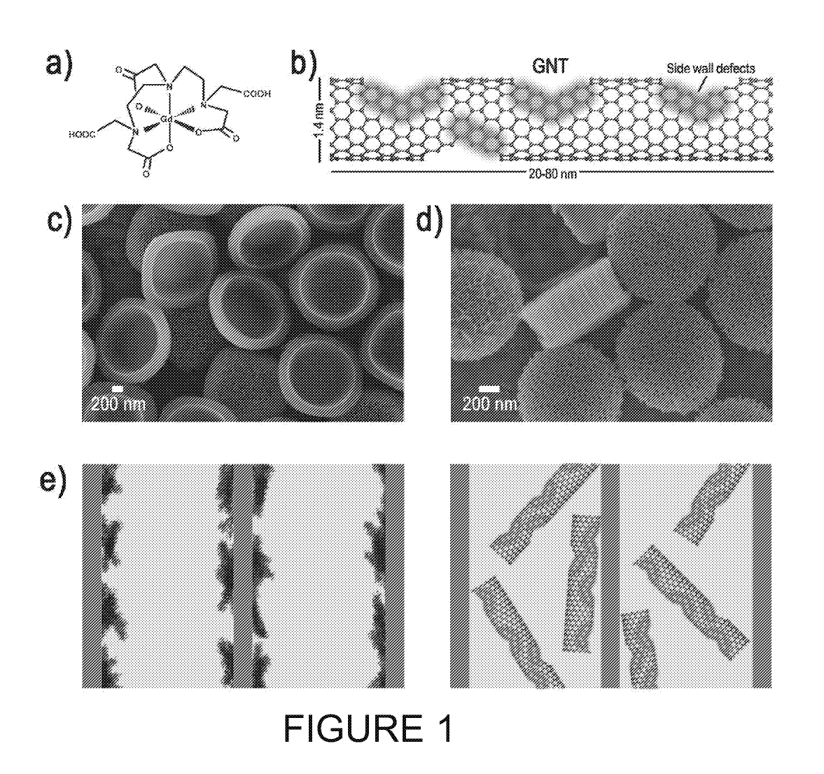

Magnevist (Gd-DTPA) is an example of gadolinium polyamino carboxylate complexes (FIG. 1A), widely used in the clinic as T₁-weighted MRI contrast agents⁶. GNTs are carbon nanostructure-based lipophilic contrast agents showing great promise in MRI. As shown in FIG. 1B, GNTs are nanoscale carbon capsules (derived from full-length single-walled carbon nanotubes) with a length of 20-80 nm and a diameter of about 1.4 nm, which are internally loaded with Gd³⁺ ion clusters. Within the GNTs, the Gd³⁺ ions are present in the form of clusters (<10 Gd³⁺ ions per cluster), and each GNT contains approximately 50 to 100 Gd³⁺ ions. The Gd³⁺ clusters are stable, and the Gd³⁺ ions do not leak from the nanocapsules under physiological conditions. Because of the hydrophobic nature of their external carbon sheath, the GNTs naturally exist in the form of bundles. However, in this work, in order to achieve a more homogenous dispersion and to reduce potential toxicity, debundled GNTs were prepared and studied¹¹.

The SiMPs were microfabricated using a combination of photolithography and electrochemical etching that allows for controlling the size, shape and porosity of the particles.^{8,12} The shape can be hemispherical, quasi-hemispherical and discoidal with an effective diameter ranging from 600 nm to a few microns. The diameter of the pores can be tailored, ranging from 10 nm (small pores) to 100 nm (large pores). In this work, Gd-CAs were loaded within the nanopores of quasi-hemispherical (H-SiMPs) particles, with a nominal diameter of 1.6 μm and a thickness of about 1 μm; and discoidal (D-SiMPs) particles, with a nominal diameter of 1.0 μm and thickness of about 0.4 μm (FIG. 1). The pores had an average diameter ranging between 30-40 nm for both SiMPs, being slightly larger for the discoidal compared to the quasi-hemispherical particles.

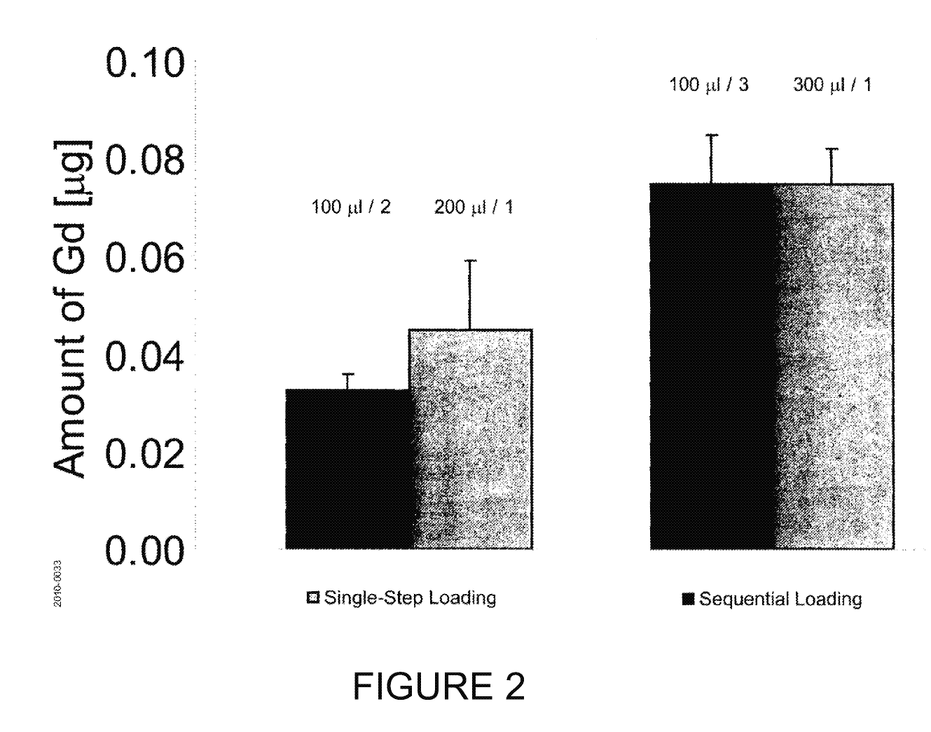

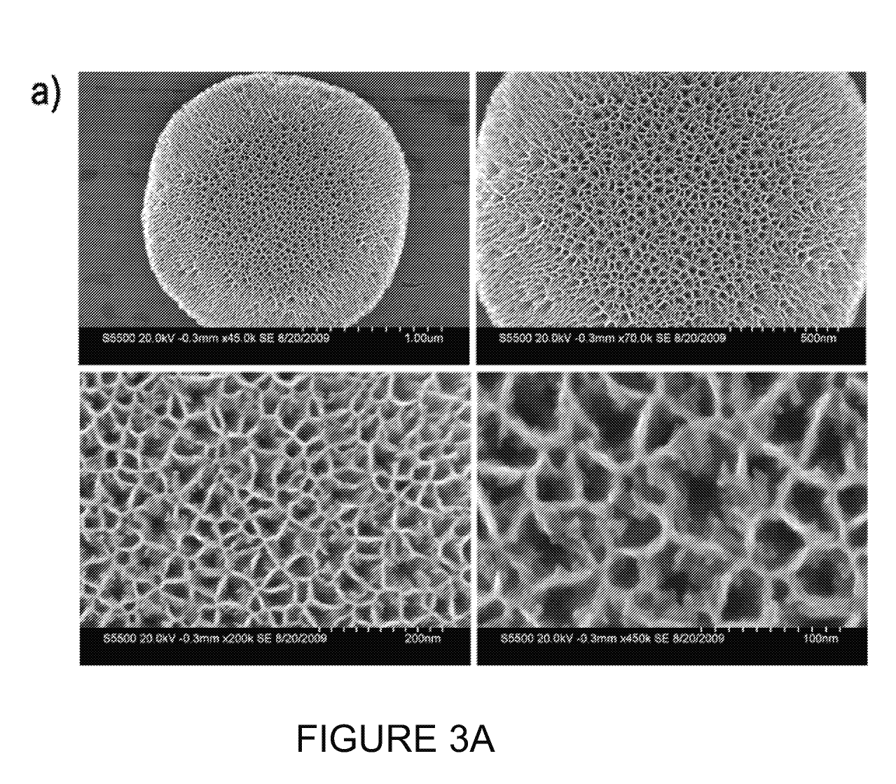

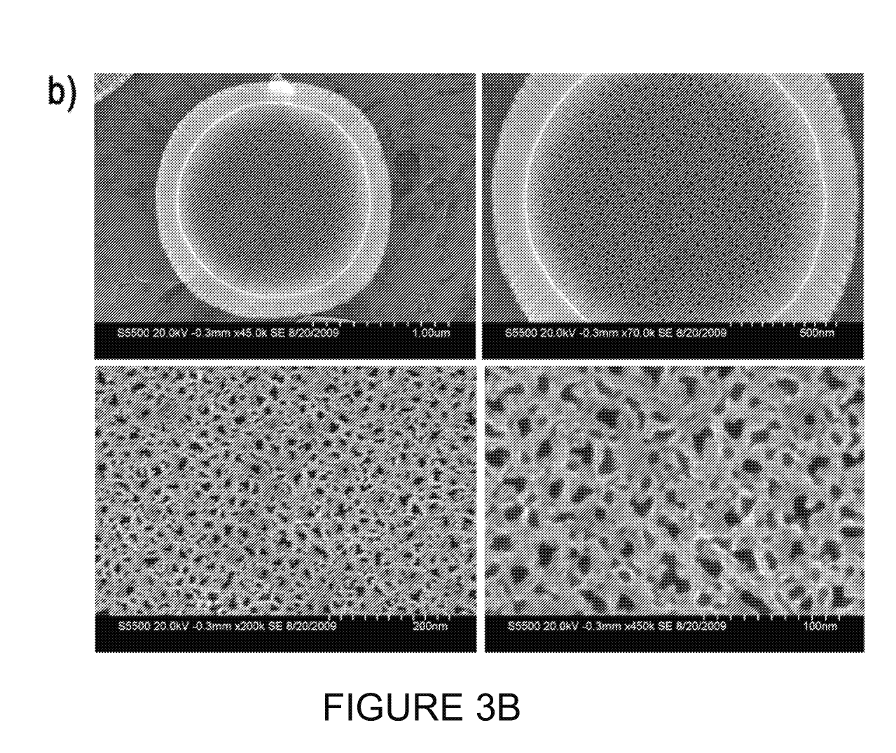

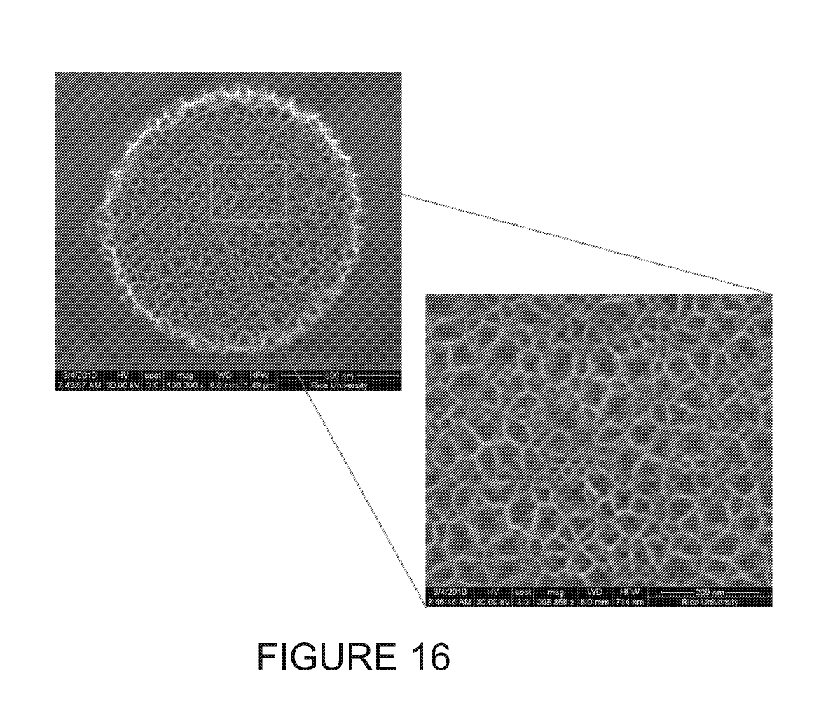

The loading and confinement of Gd-CAs within the SiMPs occur primarily through capillary forces. When the dry nanopores of the SiMPs are exposed to a concentrated aqueous solution of the CA, the latter are drawn within the pores due to capillary action. Two different loading procedures were used in this study: i) single-step loading and ii) sequential loading, where the SiMPs were exposed multiple times to the concentrated solution of Gd-CAs. For the sequential loading, the SiMPs were washed with HPLC grade water between each step. The amount of Gd³⁺ ions within the nanoconstructs was determined by elemental analysis using inductively-coupled plasma optical emission spectrometry (ICP-OES). As shown in FIG. 2 for the representative case of H-SiMP/GNT, no significant difference was observed in loading efficiency between the two procedures. However, for the single-step protocol, Gd-CAs were also seen to adhere to the outer surface of the SiMPs, whereas with the sequential loading protocol, most (if not all) of the Gd-CAs were confined within the porous structure of the SiMPs (FIG. 3—H-SiMP/GNT; FIG. 16—D-SiMP/GNT). The sequential loading process was used in this work for the preparation, characterization, and study of the nanoconstruct, despite the relative complexity.

Fabrication, Surface Modification and Characterization of Mesoporous Si Particles (SiMPs).

Hemispherical porous silicon particles with 1.6 μm diameter and discoidal porous silicon particles with 1 μm diameter and 400 nm thickness were used in the research. All the particles were fabricated in the Microelectronics Research Center at The University of Texas at Austin by combination of standard photolithography and electrochemical etching. Hemispherical particles were fabricated following protocols previously reported by our group^{8,12}.

Discoidal particles were fabricated by newly developed protocols: briefly, heavily doped p++ type (100) silicon wafers with resistivity of 0.005 ohm-cm (Silicon Quest, Inc, Santa Clara, Calif.) were used as the silicon source. A 400 nm porosity layer was formed by applying a 7 mA/cm current for 125″ in a 1:3 HF(49%):ethanol solution. The electrical current was then increased to 76 mA/cm and applied for 8″ forming a high porosity release layer. A 40 nm SiO₂ layer was deposited by Low Pressure Chemical Vapor Deposition at 400° C. Standard photolithography was used to pattern a 1 μm circular pattern with 1 μm pitch over the SiO₂ capped porous layer using a contact aligner (K. Suss MA6 mask aligner) and NR9-500P photoresist (Futurrex Franklin, N.J., USA). The pattern was transferred into the porous double layer by dry etch in CF₄ plasma (Plasmatherm 790, 25 sccm CF₄, 100 mTorr, 200W RF). The capping SiO₂ layer was removed in 49% HF, and the particles were released from the substrate by sonication in isopropanol. The particles were treated with H₂O₂ at 100° C. to oxidize the surface.

Volumetric particle size, size distribution and count were obtained using a Multisizer 4 Coulter® Particle Counter (Beckman Coulter, Fullerton, Calif., USA). Prior to the analysis, the samples were dispersed in the balanced electrolyte solution (ISOTON® II Diluent, Beckman Coulter Fullerton, Calif., USA) and sonicated for 20 seconds to ensure a homogenous dispersion. The zeta potential of the silicon particles was analyzed in phosphate buffer (PB, pH 7.3) using a ZetaPALS Zeta Potential Analyzer (Brookhaven Instruments Corporation, Holtsville, N.Y., USA). The sample cell was sonicated for 2 min before the analysis, and an electrode-probe was then put into the cell. Measurements were conducted at room temperature in triplicate. Particles structure and integrity were verified by SEM.

Fabrication and Surface Modification of Gadonanotubes

Arc discharge produced full-length, single-walled carbon nanotubes (SWNTs) were purchased from Carbolex, Inc (Broomall, Pa.). As obtained SWNTs (length>1 μm) were cut into ultra-short SWNTs (US-tubes, length 20-50 nm) by fluorination and pyrolysis.²⁹ Due to their hydrophobic nature US-tubes exist in the form of bundles. In order to obtain a homogenous dispersion, US-tubes were treated with Na⁰/THF.¹¹ This process produces mainly individual US-tubes or very small bundles (2-3 tubes). Individual US-tubes were then loaded with Gd³⁺ ions by soaking and sonication (30 C water bath sonicator) in aqueous GdCl₃ solution⁹ to produce individual gadonanotubes (GNTs). The absence of externally-adhered Gd³⁺ ions was confirmed using ICP-OES and relaxivity measurements. As produced, GNTs were then dispersed in a biocompatible, non-ionic, Pluronic® (Polyethylene oxide-polypropylene oxide block copolymer, BASF corporation, NJ) surfactant (1.0% WN) to yield a stable aqueous dispersion. The solution was centrifuged at 3200 rpm for 10 minutes and the supernatant was dialyzed against running water to remove any excess surfactant. The resulting aqueous dispersion was used for the SiMP loading experiments.

Loading of SiMPs with MAG and GNTs

For loading the pores of the Si particles with the Gd-CAs, aliquots of mesoporous Si particles were lyophilized to dryness for 6 hours in non-stick plastic tubes using Labconco® FreeZone™ Freeze Dryer system. Two protocols were tested: (1) single-step loading and (2) sequential loading. During the 1st experimental setup, dry Si particles were mixed with 300 μL of Gd-CAs (Magnevist or Gadonanotube) solution. The resulting suspension was sonicated (30 C water bath sonicator) for 5 minutes and centrifuged for 10 min at 3200 rpm. The supernatant was discarded and the sediment washed twice with deionized water to remove any excess of Gd CAs adhering to the outer surface of the Si particles. For the sequential loading experiments, the Si particles were introduced initially to 100 μl of Gd-CAs stock solutions, followed by sonication and centrifugation. After the supernatant was discarded, another 100 μl of the stock solution was added followed by sonication and centrifugation. The process was repeated with addition of another 100 μl of stock solution (total volume of the stock solution added is 300 μl) followed by loading and washing twice with DI water. In order to estimate the efficiency of loading, the particles were dissolved in 1N NaOH overnight. The resulting solution was treated with ˜26% HClO₃ and heated to dryness. The resulting precipitate was dissolved in 2% HNO₃. Si and Gd ions released from the particles during the degradation process were measured using a Perkin-Elmer Elan 9000 inductively coupled plasma-optical emission spectrometer (ICP-OES) respectively. A calibration run including the internal control (Yttrium, 5 ppm) was made before each group.

Scanning Electron Microscopy

Specimens were mounted on SEM stubs (Ted Pella, Inc.) either using conductive adhesive tape (12 mm OD PELCO Tabs, Ted Pella, Inc.) or by applying wet samples to stubs and air drying in a desiccator. Samples were sputter coated with a 10 nm layer of gold using a Plasma Sciences CrC-150 Sputtering System (Torr International, Inc.). SEM images were acquired under high vacuum, at 20.00-30.00 kV, spot size 3.5-5.0, using a FEI Quanta 400 FEG ESEM equipped with an ETD (SE) detector.

Relaxometry Studies

The 1/T₁NMRD profiles of debundled GNTs (GadoDex) were obtained at 310.0 K on a Stelar Spinmaster Fast Field-Cycling relaxometer (covering a continuous magnetic field from 2.35×10⁻⁴ to 0.47 T, proton Larmor frequencies of 0.01 to 20 MHz); on Bruker Minispecs (30, 40 and 60 MHz); and on Bruker spectrometers (100, 200 and 400 MHz). Spin-lattice relaxation (T₁ relaxation) times of GNTs in nanoporous silicon particles were measured in a Spin track bench top relaxometer (Process NMR associates, CT) operating at 60 MHz and 37° C. with a 5 mm probe. T₁-relaxation times were measured using inversion recovery sequence and HPLC grade water was used as diamagnetic control. Phantom studies in clinical scanner were performed in a 1.5 T commercial scanner (Achieva, Philips Medical Systems, Best, The Netherlands) equipped with a 32-channel radiofrequency system. A 32- or 16-element phased-array surface coil was used for MR signal reception. An inversion recovery sequence was used for image acquisition with TR=7500 ms and TE=20 ms.

Synthesis of Dextran-Coated Gadonanotubes (GadoDex)

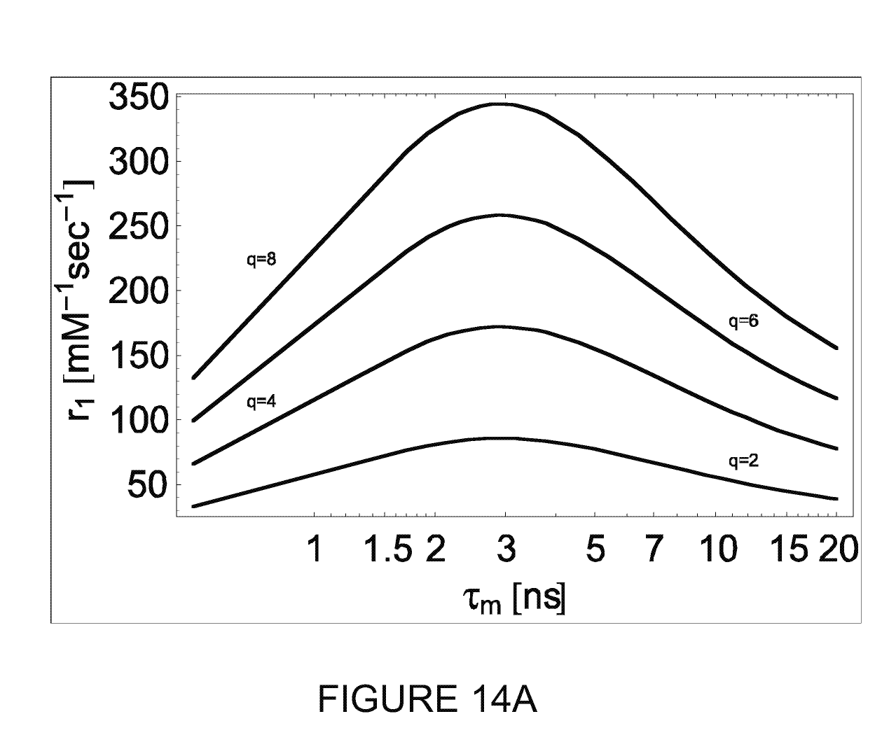

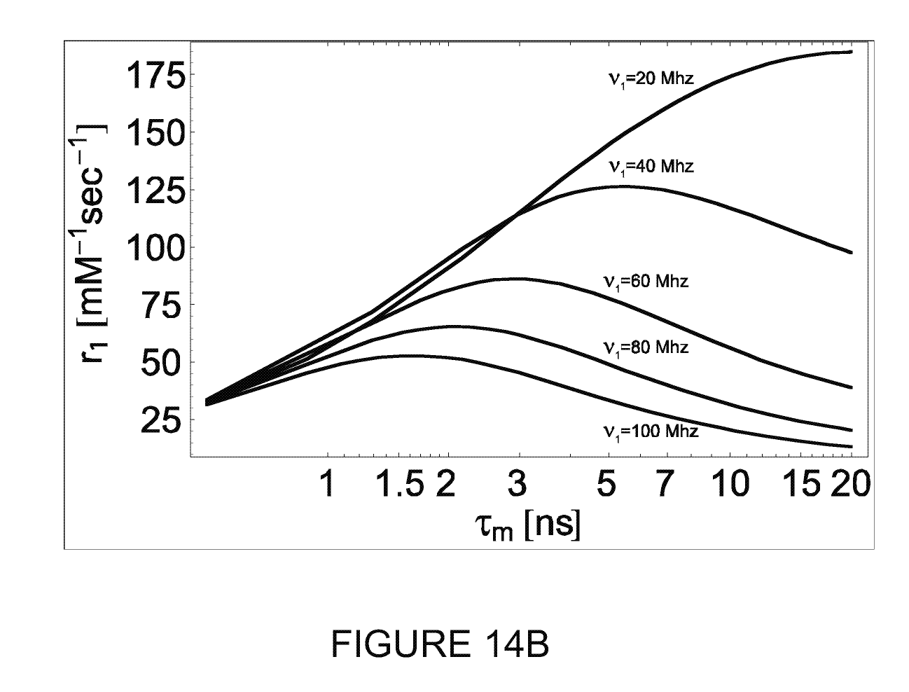

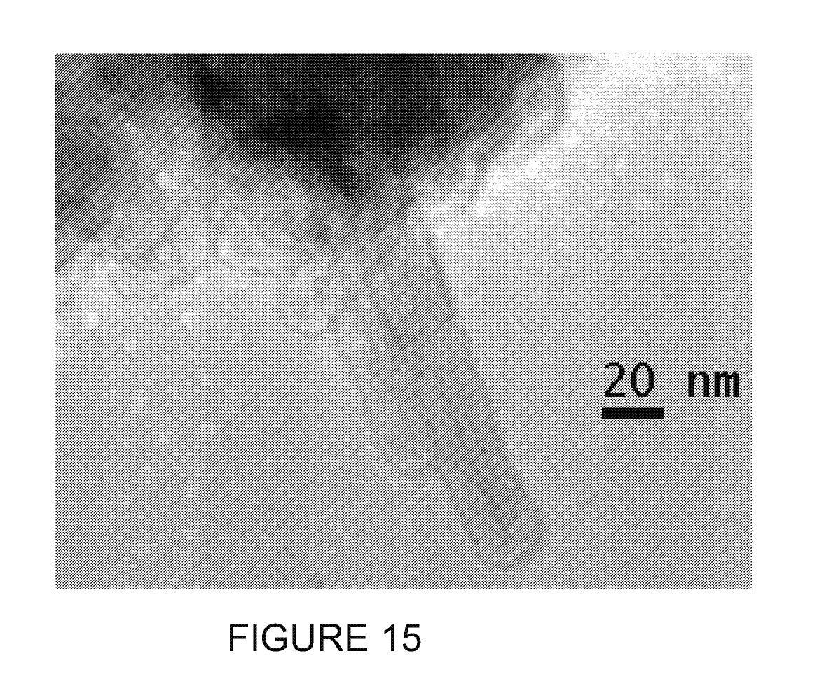

Full-length, single-walled carbon nanotubes were cut into ultra-short tubes (US-tubes) of 20-50 nm in length by fluorination followed by pyrolysis at 1000° C. in an inert Ar atmosphere.²⁹ As produced, bundled US-tubes were then reduced to form individual US-tubes using Na⁰/THF.¹¹ Dextran, with an average molecular weight of 9-11 KDa, was purchased from Sigma-Aldrich (St Louis, Mo.) and carboxylated using 40% NaOH as reported previously.³⁰ Individual US-tubes were loaded with Gd³⁺ ions as previously reported to produce individual GNTs.⁹ The individual GNTs were then refluxed with the carboxylated dextran solution for 45 minutes and then precipitated with methanol, along with cooling to room temperature. The precipitate (GadoDex; FIG. 14) was dispersed in water using probe sonication for 2 minutes and allowed to settle over night. The supernatant solution was then dialyzed against running water for 2 days using a 50 KDa MW cutoff membrane to remove any free dextran.

Statistical Analysis

All results shown are expressed as mean±standard deviation (SD). The statistical analysis of the data was carried out by Student's t test. Significance was fixed to p<0.05 and 0.1 depending on the experiments.

MRI Characterization of the Nanoconstructs

The ability of a paramagnetic material to act as an MRI contrast agent is expressed in terms of its relaxivity (rᵢ). This can be described as the change in the relaxation rate (1/Tᵢ; s⁻¹) of water protons per mM concentration of the CAs and can be calculated using the expression rᵢ=(1/Tᵢ-1/T_{id})/[CA], where Tᵢ is the relaxation time in the presence of the CA, T_{id} is the relaxation time in the absence of CA, and [CA] is the concentration of the Gd³⁺ ion present in solution (mM).

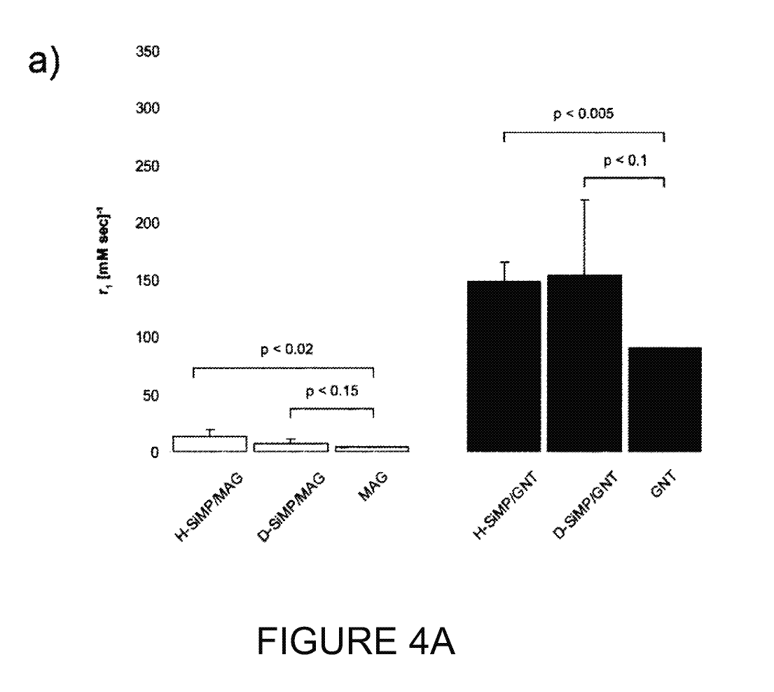

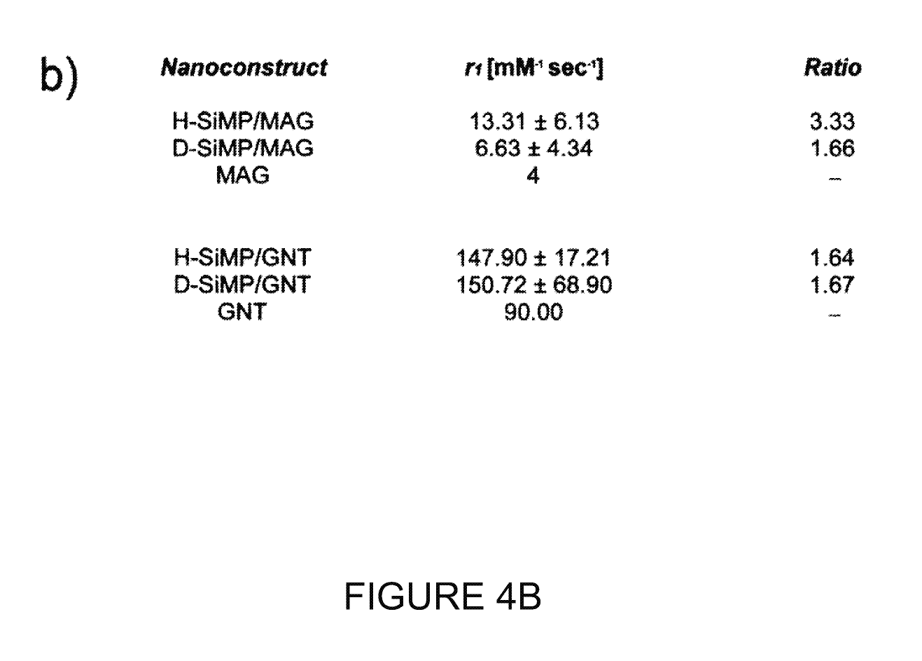

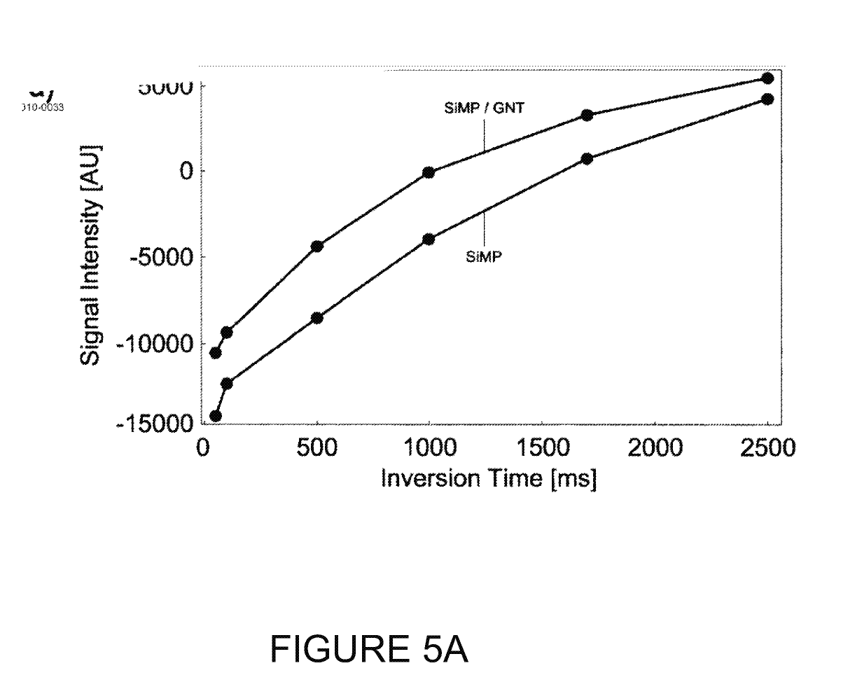

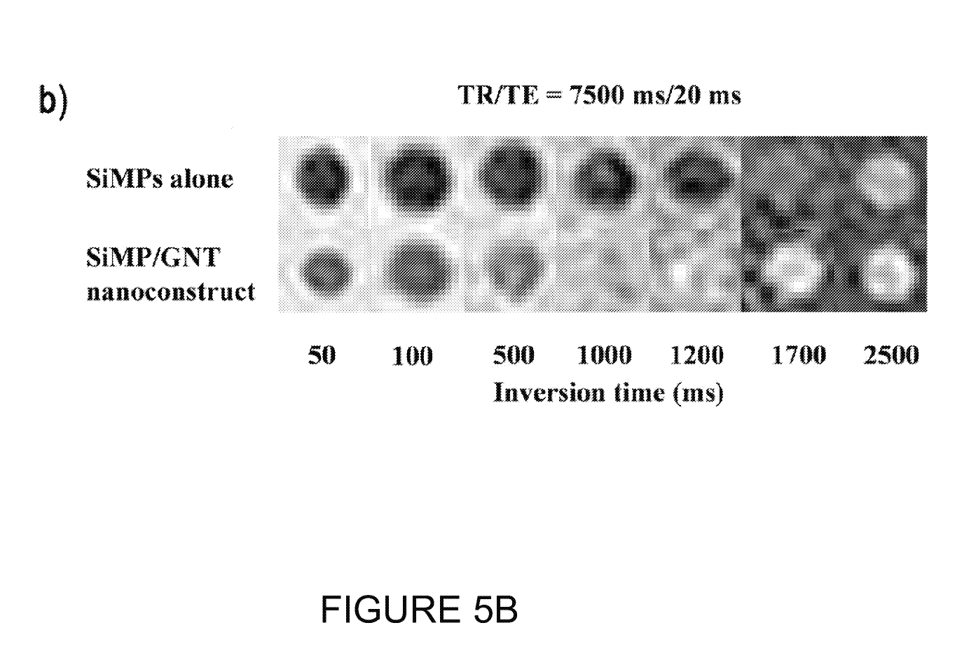

The loaded SiMPs were examined for their longitudinal relaxation properties using a benchtop relaxometer at 1.41 T and 37° C. (Bruker Minispec mq-60). The longitudinal relaxation time (T₁) was determined using an inversion recovery pulse sequence. Empty SiMPs alone showed no contrast enhancement. The longitudinal relaxivity, r₁, measured for the four different nanoconstructs is presented in FIG. 4. A statistically significant increase in r₁ was observed for all nanoconstructs, compared to the Gd-CA alone: for MAG, r₁ increased by about 3 times with the H-SiMP and 1.5 times with the D-SiMP; for GNTs, r₁ increased by about 1.5 times for both SiMPs. Compared to naked (aqueous) Gd³⁺ ion (r₁˜8 mM⁻¹ s⁻¹) and to the clinically-used Gd-based CAs (r₁˜4 mM⁻¹ s⁻¹), the longitudinal relaxivity of the nanoconstructs is about 18 and 40 times larger, respectively. Also, with MAG, the H-SiMPs performed slightly better than the D-SiMPs. This could be attributed to the high water solubility of MAG and the slightly larger average pore size of the D-SiMPs. For the representative case of the H-SiMP/GNT nanoconstruct, the contrast enhancement properties were also examined using a clinical MRI scanner at 1.5 T, and the results are presented in FIG. 5. The H-SiMP/GNT construct showed a significantly lower inversion time (Tᵢₙᵥ=˜1200 ms) compared to empty SiMP (Tᵢₙᵥ=˜1700 ms) (FIG. 5b), demonstrating that the contrast enhancement efficacy is due to the GNTs within the H-SiMP/GNT nanoconstruct.

Longitudinal Relaxivity and Geometrical Confinement

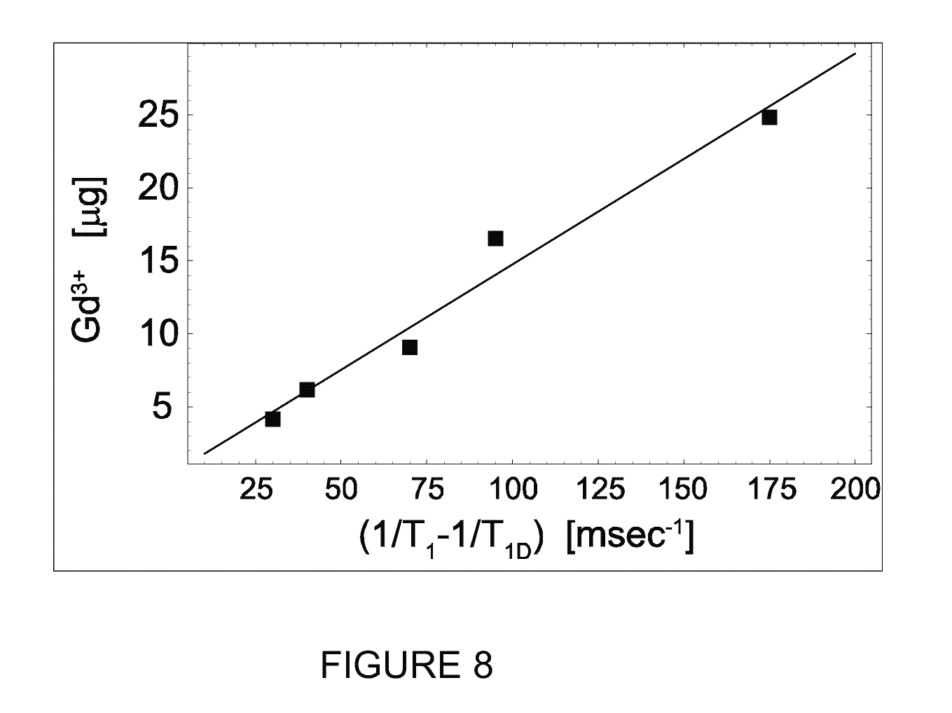

The classical theory for predicting the efficiency of MRI CAs is based on the work of Solomon, Bloembergen and Morgan², which is especially applicable in the medium-to-high-field regime (>0.1 T) (also referred to as the SBM theory, See Example 2). In this approach, the longitudinal relaxivity r₁ comprises two contributions: the inner-sphere relaxivity r₁^{IS} and the outer-sphere relaxivity r₁^{OS}. For r₁^{IS}, the most influential parameters are (i) the number, q, of fast-exchanging water molecules within the inner-sphere; (ii) the characteristic tumbling time, τ_{R}, of the agent together with its inner-sphere water molecules; (iii) the characteristic water proton residence lifetime, τₘ, of the inner-sphere water molecules; and (iv) the separation distance, r_{GdH}, between the water protons and the metal ion. In r₁^{OS}, arising from the translational diffusion of water molecules near the Gd³⁺ ions, the most influential parameter is the diffusion correlation time τ_{D}(Example 2). Whilst for MAG, the inner- and outer-sphere contribute almost equally to the longitudinal relaxivity r₁ (FIG. 8); for GNTs, as detailed later, the major contribution comes from the inner-sphere relaxivity.

Most research devoted to the design of new, high-efficiency CAs has focused on controlling the above parameters with the aim of optimizing the relaxivity. This has lead to the development of a variety of nanoparticle-based CA constructs.¹⁴ These include, for example, paramagnetic liposomes obtained by loading Gd³⁺-ion-containing amphiphilic lipid into the bilayer membrane¹⁵ (r₁=11 mM⁻¹ s⁻¹ at 25 MHz; Gd-DOPC in Table 1). Also, a dendrimer-based nanoprobe,¹⁶ with Gd³⁺-ion-chelates covalently attached to PAMAM dendrimers, have shown r₁=20 mM⁻¹ s⁻¹ at 130 MHz. By engineering the bond between Gd³⁺ ions and surrounding molecules, with the aim of increasing the rate of water exchange (reduce τₘ) and the characteristic tumbling time, τ_{R}, Gd³⁺-ion complexes bound to humans serum albumin (GdL1-HSA complex in Table 1) have demonstrated r₁ up to 130 mM⁻¹ s⁻¹ at 20 MHz ¹⁷, and even larger values, up to 130 mM⁻¹ s⁻¹ at 65 MHz, have been obtained for engineered proteins chelated with Gd³⁺-ions (Gd+3-Ca3.CD2 in Table 1)¹⁸.

Also, non-covalent functionalization of carbon nanotubes with amphiphilic Gd³⁺ chelates have been studied recently¹⁹ showing large r₁'s, up to 50 mM⁻¹ s⁻¹ at 20 MHz. Finally, water-soluble gadofullerenes^{20,21} and GNTs⁹ are known to display relaxivities as large as r₁˜40 mM⁻¹ s⁻¹ at 20 MHz for the gadofullerenes and ˜170 mM⁻¹ s⁻¹ for bundled GNTs. In this work, a completely different and more general approach is proposed for enhancing the r₁ relaxivity of Gd-CAs: the characteristic parameters q, τ_{R}, τₘ and τ_{D} could be modified by confining the agent within a nanoporous matrix.

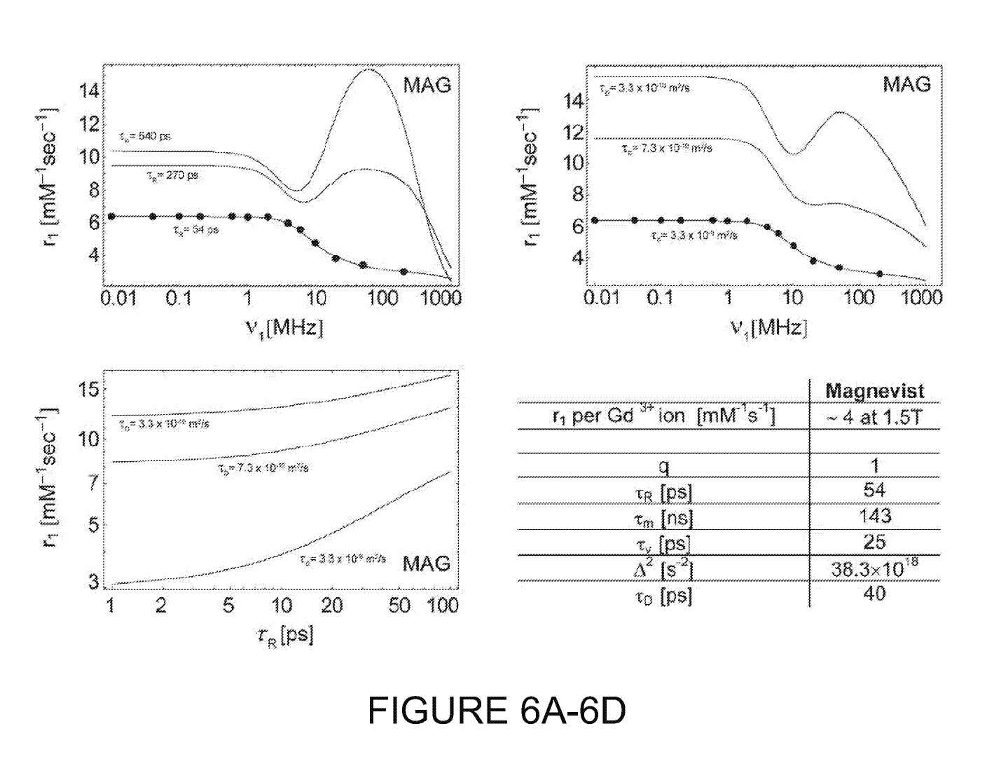

To interpret the boost in relaxivity, it is useful to analyze how the governing parameters listed above would affect the r₁ starting from the Gd-CAs alone. For MAG, the inner- and outer-sphere contribute almost equally to the longitudinal relaxivity (Example 2), generating a r₁˜4 mM⁻¹ s⁻¹ at 1.5 T. The effect of confining these molecules into nanopores is twofold: (1) to reduce the ability to freely rotate, thus increasing the characteristic tumbling time τ_{R}(inner-sphere); and (2) to reduce the mobility of the outer-sphere water molecules, thus increasing the correlation time τ_{D}(outer-sphere). FIGS. 6A-B shows that r₁ grows with τ_{R}(=54, 270 and 540 ps) and τ_{D}(=40, 180 and 400 ps), reaching at 1.5 T values close to 14 mM⁻¹ s⁻¹ measured for the H-SiMP/MAG. The inner- and outer-sphere contributions would be again equally important, as demonstrated in FIG. 6C.

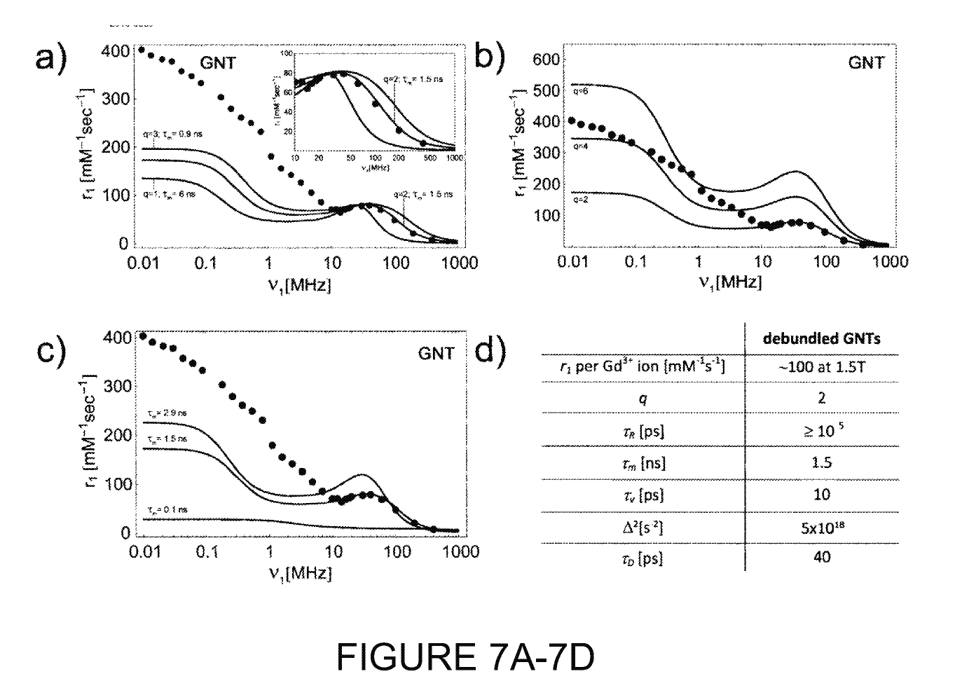

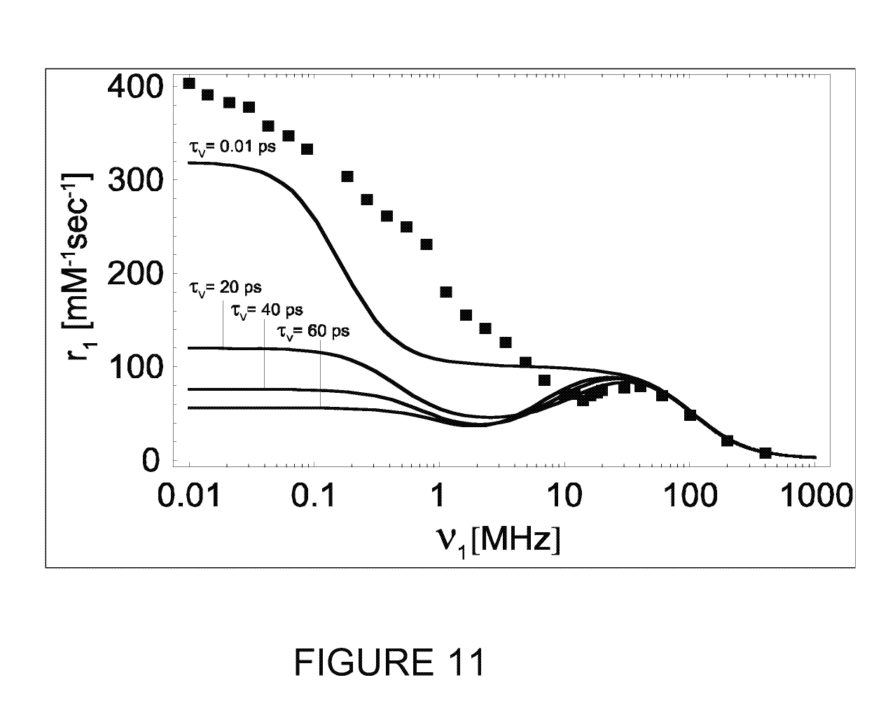

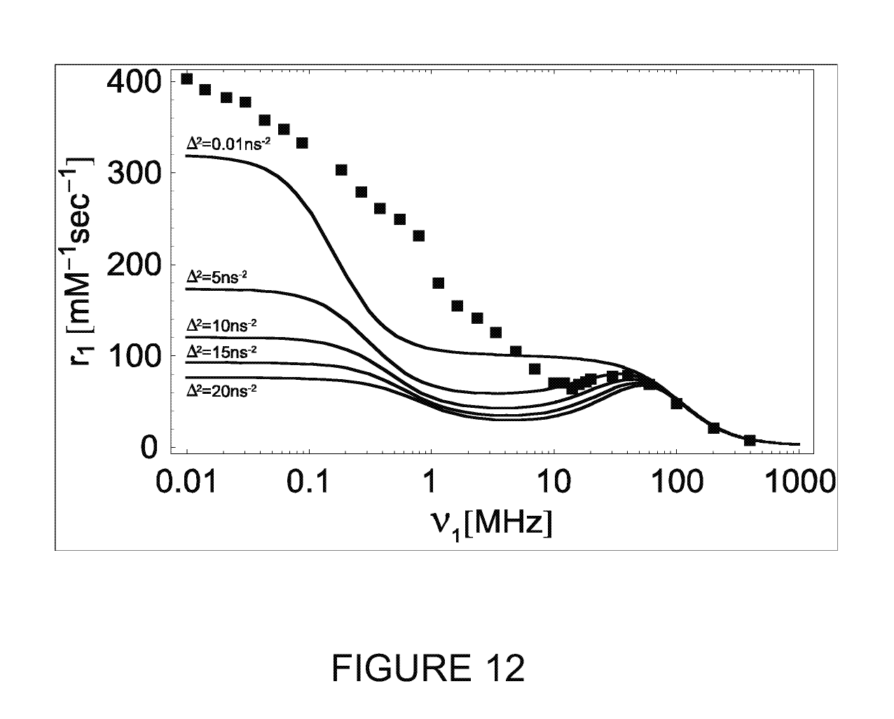

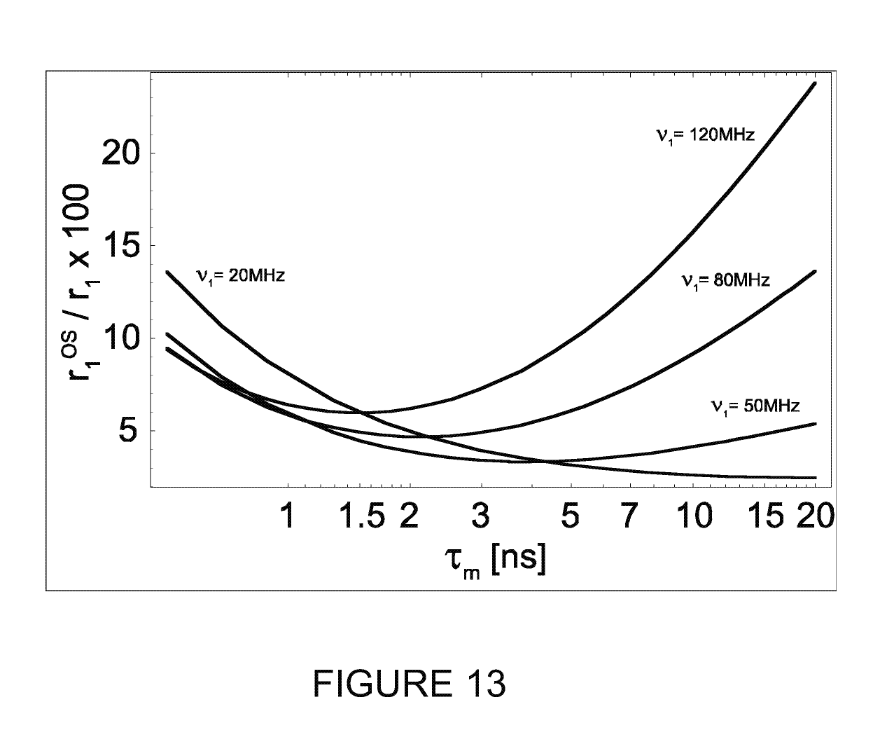

The experimental NMRD profile (solid dots) for the debundled GNTs (solubilized in water with a dextran coating, i.e. GadoDex, see Example 2) is shown in FIG. 7A together with three solid lines representing the best fit of the experimental data from the SBM Theory (Example 2). The theoretical predictions can accurately reproduce the experimental NMRD profile only in the medium-to-high field regime (ν_{I}>10 MHz), which is the clinically-relevant range. The best fit was obtained for q=2, τ_{R}=100 ns, τₘ=1.5 ns, r_{GdH}=0.31 nm, with the values for other parameters as listed in the table of FIG. 7. The accuracy of the fitting is clearly shown in the inset of FIG. 7A. The characteristic τ_{R} value used is relatively long when compared to other Gd-based CAs so far studied (Table 1). However, it should be noted that for a spherical CA of radius a=5 nm tumbling in aqueous solution, the characteristic τ_{R} is about 100 ns, and larger values can be estimated for a cylindrical nanoparticle (GNTs, 20-80 nm long and 1.4 nm in diameter) tumbling within a SiMP nanopore (Example 2). As regarding the other fitting parameters (except for q, τ_{R} and τₘ as discussed above), their values fall within the ranges normally observed for Gd-based CAs (Table 1).² In addition, the simulated NMRD profiles have been observed to be quite insensitive to variations in τ_{ν} and Δ² (FIGS. 11 and 12). Also, for the GNTs, it has been estimated that the inner-sphere contribution to r₁ relaxivity dominates the outer-sphere contribution (r₁^{OS}/r₁^{IS}<1) (FIG. 13), in agreement with recent findings which confirm that this is generally the case with slowly rotating compounds with large relaxivity.²²

The nanoconstruct obtained by loading debundled GNTs (pluronic surfactant) into the SiMPs (quasi-hemispherical and discoidal) demonstrated a r₁ relaxivity of ˜150 mM⁻¹ s⁻¹ at 1.41 T, which is significantly larger than the ˜90 mM⁻¹ s⁻¹ observed for the debundled GNTs before loading. Starting from the fitting parameters obtained for the debundled GNTs alone, the theoretical NMRD profiles for different values of q, namely, q=2, 4 and 6 (fixed τₘ=1.5 ns), and for different values of τₘ, namely, τₘ=0.1, 1.5 and 2.9 ns (fixed q=2), are plotted in FIGS. 7B and 7C, respectively. Given the large value of τ_{R} for GNTs, its contribution to r₁ is minor compared to the other two parameters. Increasing τₘ at constant q leads to significant narrowing of the relaxivity peak and also the shifting of the peak to lower field strengths (FIG. 7C). In addition, it was observed that, in the medium-to-high-field regime (ν_{I}>10 MHz), a greater increase in r₁ can be achieved by increasing q than by increasing τₘ(FIGS. 7B-7C).

The relaxation time measurements clearly demonstrate that the SiMPs themselves do not contribute to the relaxivity of the nanoconstruct. Thus, the increase in relaxivity observed for the nanoconstruct can be attributed solely to the geometrical confinement of the debundled GNTs and their final organization within the pores. The confinement of debundled GNTs within the SiMPs nanopores could increase their tumbling time, τ_{R}, to an even greater extent because of the contact with the pore walls and the increase in effective viscosity of the aqueous solution trapped within the pores. Although the surfactant wrapping would prevent the aggregation of debundled GNTs within the pores, the debundled GNTs, packed in close proximity to one another within the nanoconstruct would resemble a uniform nanotube bundle, and indeed, the r₁ relaxivity of the nanoconstruct (˜150 mM⁻¹ s⁻¹ per Gd³⁺ ion) is similar to the value reported for bundled GNTs (r₁˜170 mM⁻¹ s⁻¹) ⁹. Pseudo-aggregation of debundled GNTs inside SiMPs could result in water molecules getting trapped in the interstices of the GNT bundles. These trapped water molecules would likely diffuse slowly to the bulk water, which could increase the proton exchange rate. The diffusion rate of these confined water molecules to the bulk would be slower than translational diffusion of bulk water protons, and confined water molecules with a slow diffusion rate have previously been shown to increase the relaxivity of aggregated gadofullerenes.^{20,21}

Conclusions

A boost in MRI was demonstrated upon geometrical confinement of two different GdCAs into the nanoporous structure of microfabricated particles. Geometrical confinement can reduce the ability of CAs to tumble; decrease the mobility of the water molecules; favor clustering and mutual interactions among the loaded CAs thus altering the original values of the governing parameters q, τₘ, τ_{R} and τ_{D}, and potentially others. Not only do the delivery systems developed in this work exhibit high r₁ values, but also constitute a formidable particle-based system for efficient intravascular delivery. The size, shape and surface properties of the SiMPs can be rationally designed^{23,24} and tailored¹² to enhance the accumulation of Gd-CAs within the biological target site²⁵; to alter overall half-life in blood and to control degradation.²⁶ These delivery systems could also play an important role in the development of single-cell imaging techniques, where high relaxivity (r₁>100 mM⁻¹ s⁻¹) and large localized Gd³⁺ concentration ([Gd³⁺]>10⁷/cell) are needed²⁷. Finally these delivery systems could be loaded with multiple agents, such as other nanoparticles, small molecules and drugs²⁸, to originate highly-multifunctional systems with imaging and therapeutic capabilities.

### Example 2

### Supplemental Information for Example 1

Mathematical Model for Estimating the Longitudinal Relaxivity of MRI CAs

The ability of MRI CAs to shorten the longitudinal T₁ relaxation time of water (T₁=3000 ms) is reflected by their r₁ relaxivity. This is given by summing the contributions of the so-called inner-sphere r^{IS} and outer-sphere r^{OS} relaxivities. The inner-sphere contribution comes from the nuclear spin residing in the water molecules entering the first coordination shell of the metal ion of interest, whilst the water molecules outside of this shell contribute to the outer-sphere portion. The classical theory for estimating the inner-sphere contribution is due to the work of the SBM theory.³ This simplified theory provides a close form expression for r^{IS} and can be effectively used to interpret experimental data on the relaxivity of several complexes. In particular it has been shown that the SBM theory can accurately predict and reproduce NMRD profiles in the high field regime (B>0.25 T) which are of clinical relevance for MRI technology.³

For the inner-sphere contribution, the longitudinal relaxivity rₗ^{IS} is given as:

$${r}_{1}^{IS} = \frac{{P}_{M}}{{c}_{Gd}} \frac{q}{{T}_{1 m} + {τ}_{m}} 1.8 \times {10}^{- 5} \frac{q}{{T}_{1 m} + {τ}_{m}} \qquad ( SI \text{-1)}$$

In this formula, c is the concentration of metal ions in solution (in mM) and P_{M} is the mole fraction of the metal ions¹¹; q denotes the number of fast exchanging water molecules in the first hydration shell of a paramagnetic metal ion (inner sphere water molecules); T₁ₘ and τₘ are respectively the spin-lattice relaxation time and residence lifetime of the above inner sphere water molecules. Whilst τₘ is an intrinsic property of the complex, the characteristic time τ₁ₘ can be expressed through the Solomon-Bloembergen relation as:

$$\frac{1}{{T}_{1 m}} = \frac{2}{15} \frac{{C}_{DD}}{{r}_{GdH}^{6}} [ \frac{7 {τ}_{c 2}}{1 + {ω}_{S}^{2} {τ}_{c 2}^{2}} + \frac{3 {τ}_{c 1}}{1 + {ω}_{1}^{2} {τ}_{c 1}^{2}} ] + \frac{2}{3} S ( S + 1 ) {( \frac{A}{ℏ} )}^{2} [ \frac{{τ}_{2}}{1 + {ω}_{s}^{2} {τ}_{c 2}^{2}} ] \qquad SI \text{-} 2$$

with the constant C_{DD} given by:

$$\begin{matrix}{C}_{DD} = {γ}_{1}^{2} {g}^{2} {{μ}_{B}^{2} ( \frac{{μ}_{o}}{4 π} )}^{2} S ( S + 1 ) & SI \text{-} 6 \\ {}^{1} The mole fraction {P}_{M} is given as \frac{{m}_{Gd}}{{m}_{Gd} + {m}_{{H}_{2} O}} \approx \frac{{m}_{Gd}}{{m}_{{H}_{2} O}} & SI \text{-} 3\end{matrix}$$

where m_{Gd} and m_{H2O} are respectively the number of moles for the metal ions (Gd³⁺) and water (H₂O) in one liter of solution. For the water molecules m_{H20}=55.56 (55.56 M), whereas for the metal ions with a concentration [Gd]=0.044 mM, it follows m_{Gd}=44×10⁻⁶ M. Therefore for the present case

$$\begin{matrix}{P}_{M} \approx \frac{44 \times 1 {-}^{- 6}}{55.56} & SI \text{-} 4 \\ and the ratio {P}_{M} / [ Gd ] would be & \\ \frac{{P}_{M}}{[ Gd ]} \approx \frac{1}{44 \times {10}^{- 3}} \frac{44 \times {10}^{- 6}}{55.56} = \frac{{10}^{- 3}}{55.56} = 1.8 \times {10}^{- 5} & SI \text{-} 5\end{matrix}$$

In this formula, γ_{I} is the gyromagnetic constant for protons (γ_{I}=2.675×10⁺⁸ T⁻¹ s⁻¹); g is the electronic g-factor (g=2); S is the total electron spin of the material ion (S=7/2 for Gd³⁺); is the Bohr magneton (μ_{B}=9.274×10⁻²⁴ JT⁻¹); μ_{O} is the permeability of vacuum (μ_{O}=1.257×10⁻⁶ NA⁻²); r_{GdH} is the distance between the proton and the metal ion (r_{GdH}=0.31 nm); ωₛ and ω_{I} are the angular electronic and proton Larmor frequencies (ωₛ=658ω_{I} and ω_{I}=γ_{I}=γ_{I}B where B is the magnetic field), A is the hyperfine coupling constant (in J) and is the reduced Planck constant (=h/(2π)=1.054×10⁻³⁴ Js).

The correlation times τ_{c1}, τ_{c2} and τₑ are defined as:

τ_{d}=(τ_{R}⁻¹τₘ⁻¹+Tᵢₑ⁻¹) SI-7

In this formula, the time τ_{R} is the tumbling time of the entire metal ion and inner sphere water molecule assembly, whereas the times Tᵢₑ are defined as:

$$\frac{1}{{T}_{ie}} = \frac{1}{25} {Δ}^{2} {τ}_{v} [ 4 S ( S + 1 ) - 3 ] [ \frac{1}{1 + {ω}_{s}^{2} {τ}_{v}^{2}} + \frac{4}{1 + 4 {ω}_{s}^{2} {τ}_{v}^{2}} ] \\ \frac{1}{{T}_{2 c}} = \frac{1}{25} {Δ}^{2} {τ}_{v} [ 4 S ( S + 1 ) - 3 ] [ \frac{5}{1 + {ω}_{s}^{2} {τ}_{v}^{2}} + \frac{2}{1 + 4 {ω}_{x}^{2} {τ}_{v}^{2}} + 3 ] \qquad SI \text{-} 8$$

where Δ² is the mean square zero field splitting (ZFS) energy and τ_{ν} is the correlation time for splitting.

The electronic ωₛ and proton ω_{I} Larmor angular frequencies can be rephrased in the terms of the proton Larmor frequency ν_{I} as ω_{I}=2πν_{I} and ωₛ=658ω_{I}=658(2πν_{I}). Within the range of clinical interest, the strength B of the magnetic field ranges between 0.25 and 3 T, which corresponds to ν_{I}(=γ₁B/(2π)) equal to about 10 and 130 MHz (for B=1.5 T, ν_{I}˜65 MHz).

being [Gd] expressed in mM, for a r₁ measured in mM⁻¹ s⁻¹.

For the outer-sphere contribution, the longitudinal relaxivity r^{IS} is given as:

$${r}_{1}^{OS} = \frac{1}{{c}_{Gd}} {( \frac{1}{{T}_{1}} )}_{OS} = ( \frac{32 π}{405} ) {C}_{DD} \frac{{N}_{A}}{aD} Re [ 3 j ( {ω}_{1} ) + 7 j ( {ω}_{s} ) ] \qquad SI \text{-} 9$$

In this formula, Re means real part and the complex function j(ω) is given by

$$j ( ω ) = \frac{[ 4 + {( iω {r}_{D} + \frac{{τ}_{D}}{{T}_{1 e}} )}^{\frac{1}{2}} ]}{[ \begin{matrix}4 + 4 {( iω {r}_{D} + \frac{{τ}_{D}}{{T}_{1 e}} )}^{\frac{1}{2}} + \\ \frac{16}{9} ( iω {r}_{D} + \frac{{τ}_{D}}{{T}_{1 e}} ) + \frac{4}{9} {( iω {r}_{D} + \frac{{τ}_{D}}{{T}_{1 e}} )}^{\frac{3}{3}}\end{matrix} ]} \qquad SI \text{-} 10$$

with the characteristic diffusion time τ_{D} defined as

τ_{D}=b_{GdH}²/D SI-11

D is the sum of the diffusion coefficients of water and of the complex; b_{GdH} is the distance of closest approach of the water molecules to the complex. For the outer-sphere contribution, recently a detailed analysis has shown that it can be neglected compared to the inner sphere contribution for sufficiently large fields (B>0.25 T) and for slow tumbling construct which are generally associated with large inner-sphere relaxivities. Only under these conditions the ratio r₁^{OS}/r₁^{IS} generally smaller than 0.1, otherwise, for small inner-sphere relaxivities, r₁^{OS}/r₁^{IS} could be close to unity.

NMRD Profiles of Clinically Available Gd³⁺ Ion CAs Analyzed by SBM Theory

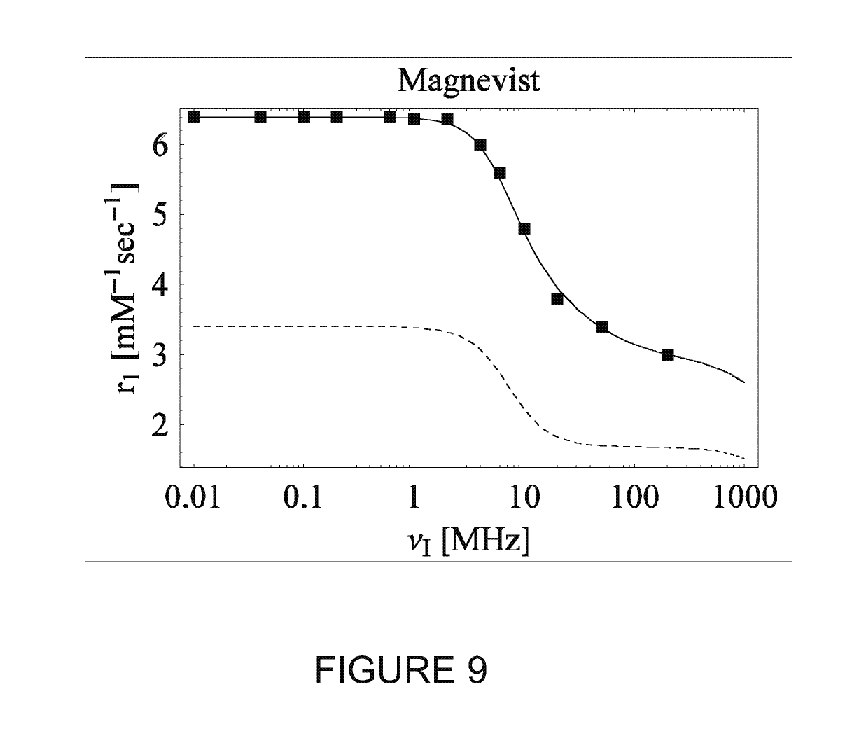

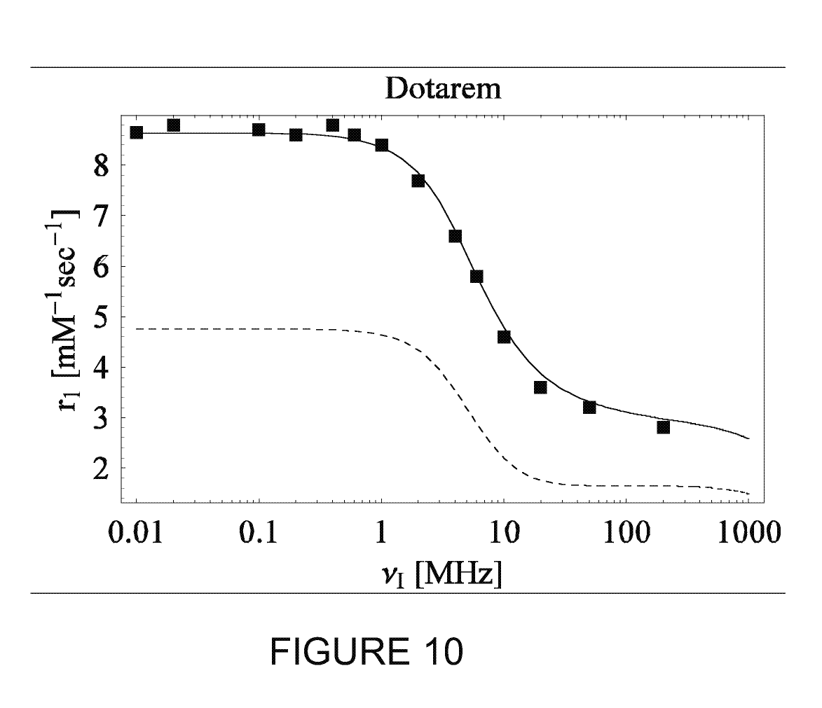

Gd-DTPA (Magenvist) and Gd-DOTA (Dotarem) are two MRI contrast agents currently used in clinical practice. Their nuclear magnetic relaxation dispersion (NMRD) profile is provided in and shown in the FIGS. 9 and 10, respectively. The dots represent the experimentally determined r₁ values at different proton Larmor frequency ν_{I}. In the same plots, the theoretical profiles derived from SBM Theory are shown: the dashed lines comprise the sole contribution of the inner-sphere (r₁^{IS}) contribution, whereas the solid lines comprise both the inner- and outer-sphere (r₁^{IS}+r₁^{OS}) contributions. Clearly, in the case of low relaxivities, the outer-sphere contribution cannot be neglected. Also, for this rather ‘classical’ Gd³⁺ CA, the SBM Theory with the addition of the outer-sphere contribution can reproduce the entire NMRD profile.

TABLE 1
Longitudinal relaxivity and related properties of Gd-based CAs.
The first six CAs are currently used in clinical settings.

| | r₁ [mM⁻¹s⁻¹] | | τ_{R} | τₘ | τᵥ | |
|---|---|---|---|---|---|---|
| Contrast Agent | per Gd³⁺ ion | q | [ps] | [ns] | [ps] | Δ² [s⁻²] |
| Gd-DTPA ¹ | 3.4 (1.41T) | 1 | 54 | 143 | 25 | 38 × 10¹⁸ |
| Gd-DOTA ¹ | 3.1 (1.41T) | 1 | 53 | 122 | 7 | 30 × 10¹⁸ |
| Gd-DTPA-BMA ¹ | 3.6 (1. 41T) | 1 | 65 | 967 | 18 | 50 × 10¹⁸ |
| Gd-HP-DO3A ¹ | 3.2 (1. 41T) | 1 | 57 | 176 | 6.5 | 12 × 10²⁰ |
| Gd-HP-DO3A ¹ | 3.2 (1. 41T) | 1 | 51 | 217 | 7.5 | 78 × 10¹⁸ |
| Gd-BOPTA ¹ | 4.1 (1. 41T) | 1 | 89 | 140 | 30 | 30 × 10¹⁸ |
| Gd-DOPC ⁶ | 11.3 (0.6T) | 1 | 1500 | 500 | 46 | — |
| GdL1-Has ⁸ | 68 (0.47T) | 1 | 6000 | 20 | 17 | — |
| Gd⁺³-CA3.CD2 ⁹ | 130 (1.5T) | 2 | — | — | — | — |
| Gd@C₆₀(OH)ₓ ¹¹ | ~40 (~1.0T) | — | 2600 | 51 | 11 | 10 × 10¹⁸ |

Rotational Diffusion Coefficients for Gadonanotubes

The following section analyzes the values taken by the various parameters for fitting the experimental data with the SBM theory. The tumbling correlation time τ_{R} can be larger than 100 ns without significantly affecting the profile. For spherical nanoparticles, the tumbling correlation time τ_{R} can be estimated with the classical formula:

$${τ}_{R} = \frac{1}{6 {D}_{r}} ; {D}_{r} = … \frac{{k}_{B} T}{8 {μπα}^{3}} \qquad SI \text{-} 12$$

leading to τ_{R}˜1 ns for a radius a=1 nm, a dynamic viscosity of the fluid μ (10⁻³ Pa·s), and a Boltzmann energy k_{B}T (=4×10⁻²¹ J). GNTs are approximately 20-80 nm long and 1.4 nm wide. The rotational correlation time for a prolate ellipsoidal particle with semi-major axis a and aspect ratio ρ=b/a can be calculated as from Ref. [20]. For a 20 nm long GNT (a=10 nm and ρ=0.07), τ_{R1}˜12 μs and τ_{R2}˜22 ns; for a 80 nm long GdNT (a=40 nm and ρ=0.0175), τ_{R1}˜1.2 ms and τ_{R2}˜87 ns.

The Effect of τ_{ν} and Δ² and Outer-Sphere Contributions on the Predicted NMRD Profile

The mathematical model presented above has been used to estimate the effect that the correlation time for splitting τ_{ν} and mean square zero field splitting energy (ZFS) can have on the NMRD profile. These are presented, respectively, in FIGS. 11-12 below, for τ=0, 20, 40, 60 ps and Δ²=0, 5, 10, 15 and 20×10⁺¹⁸ s⁻². For τ_{ν} larger than 10 ps, the profile does not change substantially within the high-field regime; whereas the effect of Δ² is somewhat more important. However, within the range of values generally observed, these parameters have a secondary effect on the NMRD profile.

The contribution of the Outer Sphere, estimated using the equations SI-9 to SI-11, is shown in FIG. 13, where the percentage ratio between r₁^{OS} and r₁ is plotted as a function of the parameter τₘ, and for different values of the field strength, ranging between ν₁=20 and 100 MHz.

Maximizing the Longitudinal Relaxivity of the Nanoconstruct Through the SBM Theory

Fixing the field strength at 60 MHz (1.41 T), the variation in r₁ with τₘ, for different values of q (parametric curves) ranging between 2 and 8, is shown in FIG. 14A. For q=6, the maximum r₁ would be about 260 mM⁻¹ s⁻¹ and for q=8 it would grow to 350 mM⁻¹ s⁻¹. Notably, the maximum relaxivity is reached for τₘ˜3 ns (=2.913 ns), independently of q. The optimal τₘ is however affected by τ_{ν} and Δ². The strength of magnetic field has also an influence on the final relaxivity of the compound as shown in FIG. 14B, where the variation in r₁ with τₘ is presented for different values of νⱼ ranging between 20 and 100 MHz (fixed q=2). The maximum in longitudinal relaxivity is predicted to reduce more than two times moving from 60 MHz (1.41 T) to 130 MHz (3 T). The optimal τₘ is however affected by τ_{ν} and Δ².

### REFERENCES

1 Mansfield, P., Snapshot magnetic resonance imaging. Angew. Chem., Int. Ed. 43 (41), 5456-5464 (2004).
2 Caravan, P., Ellison, J. J., McMurry, T. J., & Lauffer, R. B., Gadolinium(III) Chelates as MRI Contrast Agents: Structure, Dynamics, and Applications. Chem. Rev. 99 (9), 2293-2352 (1999).
3 Lauffer, R. B., Paramagnetic metal complexes as water proton relaxation agents for NMR imaging: theory and design. Chem. Rev. 87 (5), 901-927 (1987).
4 Merbach, A. E., Toth, E., & Editors, The Chemistry of Contrast Agents in Medical Magnetic Resonance Imaging. (John Wiley & Sons, West Sussex, 2001).
5 Available at http://www.imvinfo.com/
6 Laurent, S., Vander Elst, L., & Muller, R. N., Comparative study of the physicochemical properties of six clinical low molecular weight gadolinium contrast agents. Contrast Media Mol. Imaging. 1 (3), 128-137 (2006).
7 Toth, E.; Helm, L.; Merbach, A. E., Relaxivity of Gadolinium(III) Complexes: Theory and Mechanism. in The Chemistry of Contrast Agents in Medical Magnetic Resonance Imaging; Merbach, A. E.; Toth, E., Eds. (John Wiley & Sons: Chichester, 2001; pp 45-119).
8 Tasciotti, E. et al., Mesoporous silicon particles as a multistage delivery system for imaging and therapeutic applications. Nat. Nanotechnol. 3 (3), 151-157 (2008).
9 Sitharaman, B. et al., Superparamagnetic gadonanotubes are high-performance MRI contrast agents. Chem. Commun. (31), 3915-3917 (2005).
10 Hartman, K. B. et al., Gadonanotubes as Ultrasensitive pH-Smart Probes for Magnetic Resonance Imaging. Nano Lett. 8 (2), 415-419 (2008).
11 Ashcroft, J. M. et al., Functionalization of individual ultra-short single-walled carbon nanotubes. Nanotechnology 17 (20), 5033-5037 (2006).
12 Chiappini, C. et al. Tailored Porous Silicon Microparticles: Fabrication and Properties. ChemPhysChem, 2010. e-pub ahead of print
13 Li, Z. & Drazer, G., Fluid enhancement of particle transport in nanochannels. Phys. Fluids 18 (11), 117102/117101-117102/117107 (2006).
14 Mulder, W. J. M. et al., Nanoparticulate Assemblies of Amphiphiles and Diagnostically Active Materials for Multimodality Imaging. Acc. Chem. Res. 42 (7), 904-914 (2009).
15 Strijkers, G. J. et al., Relaxivity of liposomal paramagnetic MRI contrast agents. Magn. Reson. Mater. Phys., Biol. Med. 18 (4), 186-192 (2005).
16 Talanov, V. S. et al., Dendrimer-Based Nanoprobe for Dual Modality Magnetic Resonance and Fluorescence Imaging. Nano Lett. 6 (7), 1459-1463 (2006).
17 Avedano, S. et al., Maximizing the relaxivity of HSA-bound gadolinium complexes by simultaneous optimization of rotation and water exchange. Chem. Commun. (45), 4726-4728 (2007).
18 Yang, J. J. et al., Rational Design of Protein-Based MRI Contrast Agents. J. Am. Chem. Soc. 130 (29), 9260-9267 (2008).
19 Richard, C. et al., Noncovalent Functionalization of Carbon Nanotubes with Amphiphilic Gd3+ Chelates: Toward Powerful T1 and T2 MRI Contrast Agents. Nano Lett. 8 (1), 232-236 (2008).
20 Laus, S. et al. Understanding Paramagnetic Relaxation Phenomena for Water-Soluble Gadofullerenes. J. Phys. Chem. C, (111), 5633-5639 (2007).
21 Laus, S. et al. Destroying Gadofullerene Aggregates by Salt Addition in Aqueous Solution of Gd@C60(OH)x and Gd@C60[C(COOH2)]. J. Am. Chem. Soc., (127), 9368-9369 (2005).
22 Kruk, D. & Kowalewski, J., General treatment of paramagnetic relaxation enhancement associated with translational diffusion. J. Chem. Phys. 130 (17), 174104/174101-174104/174112 (2009).
23 Decuzzi, P. & Ferrari, M., The adhesive strength of non-spherical particles mediated by specific interactions. Biomaterials 27 (30), 5307-5314 (2006).
24 Decuzzi, P., Pasqualini, R., Arap, W., & Ferrari, M., Intravascular Delivery of Particulate Systems: Does Geometry Really Matter? Pharm. Res. 26 (1), 235-243 (2009).
25 Decuzzi, P. et al., Size and shape effects in the biodistribution of intravascularly injected particles. J Control Release (doi:10.1016/j.jconrel.2009.10.014, 2009).
26 Godin B., G. J. et al., Tayloring the degradation kinetics of mesoporous silicon structures through PEGylation. J. Biomedic Mat. Res. A., in print (2010).
27 Nunn, A. D., Linder, K. E., & Tweedle, M. F., Can receptors be imaged with MRI agents? Q Nucl Med 41 (2), 155-162 (1997).
28 Tanaka, T. Sustained siRNA Delivery by Mesoporous Silicon Particles. Cancer Research, in print (2010).
29 Gu, Z., Peng, H., Hauge, R. H., Smalley, R. E., & Margrave, J. L., Cutting single-wall carbon nanotubes through fluorination. Nano Lett. 2 (9), 1009-1013 (2002).
30 Kawaguchi, T. & Hasegawa, M., Structure of dextran-magnetite complex: relation between conformation of dextran chains covering core and its molecular weight. J. Mater. Sci.: Mater. Med. 11 (1), 31-35 (2000).

### Example 3

### Optimized Loading of Magnevist into Mesoporous Silicon Particles

Discoidal mesoporous silicon particles with a diameter of 1000 nm and a thickness of 400 nm were loaded with Magnevist (MAG). As set forth previously, MAG is a clinically available Gd³⁺ ion-based contrast agent for T1 weighted MRI. It has been show that by reducing the size of the pores in which MAG is loaded, the longitudinal relaxivity r1 of the nanoconstructs can be enhanced even more than what was demonstrated by using the HP particles. See Examples 1-2 and Nature Nanotechnology 5:815-821 (24 Oct. 2010).

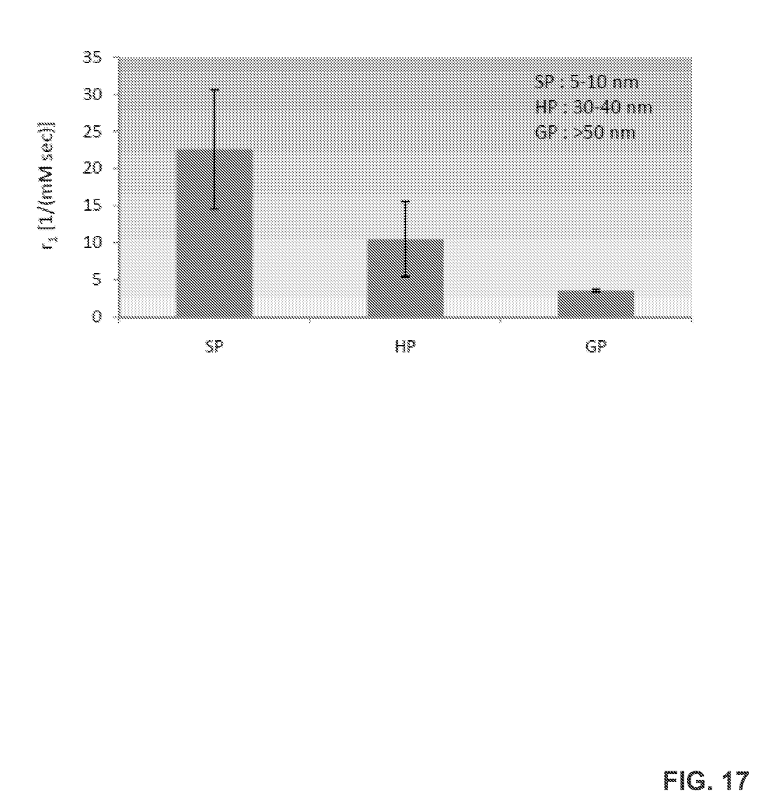

As shown in FIG. 17, a longitudinal relaxivity r1 of about 10 (mM sec)⁻¹ was demonstrated for the HP particles (pore size 30-40 nm in diameter). With the SP particles (pore size 5-10 nm in diameter), the longitudinal relaxivity r1 was boosted up to about 25 (mM sec)-1.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A composition comprising:
a) at least one porous microparticle or nanoparticle; and
b) at least one non-chelated MRI contrast agent in pores of the least one porous microparticle or nanoparticle.

2. The composition of claim 1, wherein the at least one porous microparticle or nanoparticle comprises at least one nanoporous microparticle or nanoparticle.

3. The composition of claim 1, wherein the at least one porous microparticle or nanoparticle has a pore size ranging from 5 nm to 200 nm.

4. The composition of claim 1, wherein the at least one porous microparticle or nanoparticle has a pore size ranging from 10 nm to 100 nm.

5. The composition of claim 1, wherein a maximum dimension of the at least one porous microparticle or nanoparticle is no more than 10 microns.

6. The composition of claim 1, wherein the at least one porous microparticle or nanoparticle comprises at least one non-spherical microparticle or nanoparticle.

7. The composition of claim 1, wherein at least one porous microparticle or nanoparticle comprises at least one of a hemispherical particle, a quasi-hemispherical particle, or a discoidal particle.

8. The composition of claim 1, wherein the at least one porous microparticle or nanoparticle comprises a plurality of particles that are uniform in size.

9. The composition of claim 1, further comprising a pharmaceutically acceptable carrier, wherein the at least one porous microparticle or nanoparticle is suspended in said carrier.

10. The composition of claim 1, wherein the at least one porous microparticle or nanoparticle comprises at least one silicon porous microparticle or nanoparticle.

11. The composition of claim 1, wherein the at least one porous microparticle or nanoparticle comprises at least one silica porous microparticle or nanoparticle.

12. The composition of claim 1, wherein the at least one non-chelated MRI contrast agent comprises at least one non-chelated T1 MRI contrast agent.

13. The composition of claim 12, wherein the at least one non-chelated T1 MRI contrast agent comprises at least one of Gd(III) or Mn(II).

14. The composition of claim 13, wherein the at least one non-chelated T1 MRI contrast agent comprises Gd(III).

15. The composition of claim 14, wherein the at least one non-chelated T1 MRI contrast agent comprises at least one of gadobenic acid, gadobutrol, gadocoletic acid, gadodenterate, gadodiamide, gadofosveset, gadomelitol, gadopenamide, gadopentetic acid, gadoteric acid, gadoversetamide, gadoxetic acid or a pharmaceutically acceptable salt thereof.

16. The composition of claim 15, wherein the at least one non-chelated T1 MRI contrast agent comprises gadopentetic acid or a pharmaceutically acceptable salt thereof.

17. The composition of claim 14, wherein the at least one non-chelated T1 MRI contrast agent comprises at least one carbon based particle comprising Gd(III).

18. The composition of claim 17, wherein the at least one carbon based particle comprises at least one of gadofullerene or gadonanotube.

19. The composition of claim 18, wherein the at least one carbon based particle comprises gadonanotube.

20. The composition of claim 19, wherein the at least one carbon based particle comprises unbundled gadonanotubes.

21. A method of MRI imaging, comprising:
administering to a subject in need thereof an effective amount of a composition comprising:
a) at least one porous microparticle or nanoparticle, and
b) at least one non-chelated MRI contrast agent in pores of the least one porous microparticle or nanoparticles; and
detecting a signal from the subject associated with the at least one non-chelated MRI contrast agent.

22. The method of claim 21, wherein said administering is performed intravascularly.

23. The method of claim 21, wherein the subject is a human being.

24. The method of claim 21, wherein the at least one porous microparticle or nanoparticle comprises at least one nanoporous microparticle or nanoparticle.

25. The method of claim 21, wherein the at least one porous microparticle or nanoparticle has a pore size ranging from 5 nm to 200 nm.

26. The method of claim 21, wherein the at least one porous microparticle or nanoparticle has a pore size ranging from 10 nm to 100 nm.

27. The method of claim 21, wherein a maximum dimension of the at least one porous microparticle or nanoparticle is no more than 10 microns.

28. The method of claim 21, wherein the at least one porous microparticle or nanoparticle comprises at least one non-spherical microparticle or nanoparticle.

29. The method of claim 21, wherein the at least one porous microparticle or nanoparticle comprises at least one of a hemispherical particle, a quasi-hemispherical particle, or a discoidal particle.

30. The method of claim 21, wherein the at least one porous microparticle or nanoparticle comprises a plurality of particles that are uniform in size.

31. The method of claim 21, wherein the composition further comprises a pharmaceutically acceptable carrier, and wherein the at least one porous microparticle or nanoparticle is suspended in said carrier.

32. The method of claim 21, wherein the at least one porous microparticle or nanoparticle comprises at least one silicon porous microparticle or nanoparticle.

33. The method of claim 21, wherein the at least one porous microparticle or nanoparticle comprises at least one silica porous microparticle or nanoparticle.

34. The method of claim 21, wherein the at least one non-chelated MRI contrast agent comprises at least one non-chelated T1 MRI contrast agent.

35. The method of claim 34, wherein the at least one non-chelated T1 MRI contrast agent comprises at least one of Gd(III) or Mn(II).

36. The method of claim 35, wherein the at least one non-chelated T1 MRI contrast agent comprises Gd(III).

37. The method of claim 36, wherein the at least one non-chelated T1 MRI contrast agent comprises at least one of gadobenic acid, gadobutrol, gadocoletic acid, gadodenterate, gadodiamide, gadofosveset, gadomelitol, gadopenamide, gadopentetic acid, gadoteric acid, gadoversetamide, gadoxetic acid or a pharmaceutically acceptable salt thereof.

38. The method of claim 37, wherein the at least one non-chelated T1 MRI contrast agent comprises gadopentetic acid or a pharmaceutically acceptable salt thereof.

39. The method of claim 36, wherein the at least one non-chelated T1 MRI contrast agent comprises at least one carbon based particle comprising Gd(III).

40. The method of claim 39, wherein at least one carbon based particle comprises at least one of gadofullerene or gadonanotube.

41. The method of claim 40, wherein at least one carbon based particle comprises gadonanotube.

42. The method of claim 41, wherein at least one carbon based particle comprises unbundled gadonanotube.

43. A method of making a composition for MRI imaging, comprising:
exposing at least one porous microparticle or nanoparticle to a solution comprising at least one non-chelated MRI contrast agent,
wherein the exposing causes a loading of the at least one non-chelated MRI contrast agent into pores of the at least one porous microparticle or nanoparticle.

44. The method of claim 43, further comprising drying the at least one porous microparticle or nanoparticle prior to the exposing.

45. The method of claim 43, wherein the exposing comprises:
exposing the at least one porous microparticle or nanoparticle to a first solution comprising the at least non-chelated MRI contrast agent;
washing the exposed at least one porous microparticle or nanoparticle; and
exposing the washed at least one porous microparticle or nanoparticle to a second solution, wherein the second solution also comprises the at least one non-chelated MRI contrast agent.

46. The method of claim 43, further comprising washing the loaded at least one porous microparticle or nanoparticle after said exposing.

47. The method of claim 43, further comprising sonicating said particles during said exposing.

48. The method of claim 43, wherein the at least one porous microparticle or nanoparticle comprises at least one silicon porous microparticle or nanoparticle.

49. The method of claim 48, wherein the at least one silicon porous microparticle or nanoparticle is an oxidized silicon porous microparticle or nanoparticle.

50. The method of claim 43, wherein the at least one non-chelated MRI contrast agent comprises at least one non-chelated T1 MRI contrast agent.

51. The method of claim 50, wherein the at least one non-chelated T1 MRI contrast agent comprises at least one of Gd(III) or Mn(II).

52. The method of claim 51, wherein the at least one non-chelated T1 MRI contrast agent comprises Gd(III).

53. The method of claim 52, wherein the at least one non-chelated T1 MRI contrast agent comprises at least one of gadobenic acid, gadobutrol, gadocoletic acid, gadodenterate, gadodiamide, gadofosveset, gadomelitol, gadopenamide, gadopentetic acid, gadoteric acid, gadoversetamide, gadoxetic acid or a pharmaceutically acceptable salt thereof.

54. The method of claim 53, wherein the at least one non-chelated T1 MRI contrast agent comprises gadopentetic acid or a pharmaceutically acceptable salt thereof.

55. The method of claim 52, wherein the at least one non-chelated T1 MRI contrast agent comprises at least one carbon based particle comprising Gd(III).

56. The method of claim 55, wherein the at least one carbon based particle comprises at least one of gadofullerene or gadonanotube.

57. The method of claim 56, wherein the at least one carbon based particle comprises gadonanotube.

58. The method of claim 57, wherein at least one carbon based particle comprises unbundled gadonanotubes.
